(19) United States
(12) Patent Application Publication
(10) Pub. No.: US 2013/0336940 A1
(43) Pub. Date: Dec. 19, 2013

(54) MODIFIED PIG ISLETS FOR DIABETES TREATMENT

(71) Applicants: Denis Dufrane, Wavre (BE); Sophie Veriter, Rixensart (BE); Pierre Gianello, Rixensart (BE)

(72) Inventors: Denis Dufrane, Wavre (BE); Sophie Veriter, Rixensart (BE); Pierre Gianello, Rixensart (BE)

(73) Assignee: UNIVERSITE CATHOLIQUE DE LOUVAIN, Louvain la Neuve (BE)

(21) Appl. No.: 14/001,468

(22) PCT Filed: Feb. 23, 2012
(86) PCT No.: PCT/EP2012/053060

§ 371 Date: Aug. 23, 2013

(30) Foreign Application Priority Data
Feb. 23, 2011 (EP) 11155538.9
Apr. 15, 2011 (EP) 11162588.5

(51) Int. Cl. A61K 35/39; C07K 14/605; C12N 5/071

(52) U.S. Cl. CPC A61K 35/39; C12N 5/0676; C07K 14/605
USPC 424/93.21; 435/325; 435/455; 514/44 R; 800/17

(57) ABSTRACT

The present invention relates to a modified pig islet capable of producing higher levels of glucagon than a native pig islet or capable of producing a glucagon analog, and methods for obtaining thereof. The invention also relates to a method for treating Diabetes Mellitus, and/or for regulating blood glucose levels in a subject in need thereof, comprising the administration of the modified pig islets of the invention.

### FIELD OF INVENTION

The present invention relates to the domain of the treatment of Diabetes. The present invention particularly relates to the treatment of Diabetes by transplantation of islet of Langherans from Pig.

### BACKGROUND OF INVENTION

Type I diabetes mellitus, also referred to as insulin-dependent diabetes mellitus (IDDM) or juvenile diabetes, is a chronic disease. The main symptom is a glycemia higher than normal, resulting from the failure of beta cells of the islets of Langerhans to produce insulin. In a vast majority of patients, the beta cells are destroyed by a T-cell mediated autoimmune attack.

Usual treatments consist in daily injections of insulin, in order to compensate the deficit of production by the pancreas. However, while life-saving, treatment with insulin often does not provide sufficient control of blood glucose to prevent life-shortening complications of the disease. Other treatments are thus currently in research and development. They are based on pancreas, or islets of Langerhans, transplantation, to restore a production of insulin at a normal level. Allotransplantations (transplantation of organs from human origin to humans) are limited by the shortage of human islet tissues, as well as by the need for several pancreases for each recipient. There is thus a need for alternative sources of insulin-producing cells.

Pig islet xenotransplantation might currently represent the most appropriate solution, since: (1) the supply of pig cells can be readily expanded by optimizing the supply of donor animals, (2) pig and human insulin have close structural similarities, (3) physiological glucose levels in pigs are similar to those in humans, and (4) genetic modifications of pig cells are technically possible and should solve several problems related to discordant islet xenotransplantation, for example by minimizing both the number of required islets and the risk of thrombosis (Dufrane & Gianello, Transplantation, 2008, 86(6), 753:60). WO2002/32437 describes a method of preparing porcine islets capable of producing insulin within a mammalian host.

Xenotransplantation raises the problem of immunologic response directed to the foreign organ. Immunosuppressive treatments should be continued during the life time of the patient, and are very constraining. WO2007/144389 and WO2010/032242 provide an alternative solution to limit the rejection of the implanted pig islets, through the encapsulation of the transplant in a membrane permeable to glucose, nutrients and insulin, but not to humoral/cellular immune components.

Several preclinical pig-to-non-human primate studies have been published during the last decade, with promising results regarding the production of insulin in the recipient (for a list, see Dufrane & Gianello, Transplantation, 2008, 86(6), 753:60). Clinical studies are current in human diabetic patients, such as, for example, with the use of the product DIABECELL®, developed by Living Cell Technologies.

However, the production of insulin by porcine beta cells in response to a glucose stimulation is weak as compared to human (see Henquin & Dufrane D, Diabetes. 2006, 55(12):3470-7; Dufrane, Diabetes Metab. 2007, 33(6):430-8; Dufrane, Transplantation 2010). As a result, the correction of blood glucose by implanted pig islets occurs within hours, which represents a drawback as the blood glucose level is usually corrected within minutes by human islets.

Consequently, this lower response to blood glucose leads to the need of transplanting a high number of pig islets to adequately correct the human glucose level, which is also a drawback of the treatment method as several pigs are currently used to transplant one patient.

There is thus a need for an improved method for treating diabetic patients through the xenotransplantation of pig islet.

The inventors provide hereafter an improved method based on the inventive concept of modifying pig islets to enhance production of insulin. Without willing to be bound to a theory, the inventors found that enhancing production of glucagon or an analog thereof by pig islets cells would activate insulin production pathway and lead to an increase in insulin secretion by pig islets, thereby solving the problem of low insulin production by pig islets.

The method of the invention thus presents the following advantages: (i) enhancing the production of insulin by the implanted pig islets, resulting in an adapted blood glucose level regulation, and (ii) decreasing the number of animals to be used for one xenotransplantation, resulting in an economical advantage.

### SUMMARY

The present invention relates to a modified pig islet capable of producing higher levels of glucagon than a native pig islet or capable of producing a glucagon analog.

According to an embodiment, the structure of the modified pig islet of the invention is modified to increase the proportion of glucagon producing cells.

According to an embodiment, the proportion of beta cells compared to the alpha cells in the modified pig islet of the invention ranges from about 2.5/1 to 5/1, preferably from 2.5/1 to 3.5/1, more preferably is about 2.5/1.

The present invention also relates to a method for obtaining the modified pig islet of the invention, wherein pigs are injected once with 10 to 150 mg/kg of the pig body of Streptozotocin, preferably with 30 to 100 mg/kg of the pig body of Streptozotocin, more preferably with 30 to 50 mg/kg of the pig body of Streptozotocin.

According to an embodiment of the invention, the modified pig islet is a transgenic pig islet, wherein the expression of a gene selected from the group comprising Glucagon, glucagon like peptide 1, PC (Protein Convertase) 1/3 and TORC Bcl-2 has been induced by transgenese.

Another object of the invention is a vector comprising the sequence in nucleotides of Glucagon, glucagon like peptide 1, PC (Protein Convertase) 1/3 or TORC Bcl-2, for use in the transgenic modification of a pig islet.

The present invention also relates to a method for obtaining the transgenic pig islet as described here above, using the vector of the invention, wherein said method is an in vitro method or an in vivo method.

Another object of the invention is a transgenic pig islet cell obtained by the method hereabove described.

Another object of the invention is a transgenic pig obtained by the in vivo method of the invention.

The present invention also relates to a device comprising the modified pig islet as described hereabove, or obtained by the methods of the invention.

Another object of the invention is a method for treating Type I Diabetes Mellitus in a subject in need thereof, comprising the administration of the modified pig islet as described hereabove, or obtained by the methods of the invention, or of the device of the invention.

Another object of the invention is a method for regulating blood glucose levels in a subject in need thereof, comprising the administration of the modified pig islet as described hereabove, or obtained by the methods of the invention, or of the device of the invention.

According to an embodiment, the subject is a mammal, preferably a primate, more preferably a human.

Another object of the invention is an isolated modified pig islet capable of producing glucagon like peptide 1 (GLP-1), or capable of producing higher levels of glucagon than a native pig islet.

In one embodiment, said isolated modified pig islet
is capable of producing GLP-1 and
is a transgenic pig islet expressing the GLP-1 gene.

In one embodiment, said isolated modified pig islet
is capable of producing higher levels of glucagon than a native pig islet and
is a transgenic pig islet overexpressing the glucagon gene.

Another object of the invention is the use of a vector comprising the nucleic acid sequence of glucagon like peptide 1 or of Glucagon for the transgenic modification of a pig islet.

Another object of the invention is a method for obtaining an isolated modified pig islet as herein above described, using the vector of the invention, wherein said method is an in vitro method or an in vivo method.

Another object of the invention is an isolated transgenic pig islet cell obtained by the method of the invention.

Another object of the invention is a transgenic pig comprising a modified pig islet as hereinabove described.

In one embodiment of the invention, the isolated modified pig islet of the invention is a pig islet wherein the structure of said pig islet is modified to increase the proportion of glucagon producing cells.

In one embodiment of the invention, the proportion of beta cells compared to the alpha cells ranges from about 2.5/1 to 5/1, preferably from 2.5/1 to 3.5/1, more preferably is about 2.5/1.

Another object of the invention is a method for obtaining the isolated modified pig islet whose structure is modified, wherein pigs are injected once with 30 to 50 mg/kg of the pig body of Streptozotocin and wherein modified pig islets are isolated 2 to 6 months after the administration of Streptozotocin.

Another object of the invention is a device comprising the isolated modified pig islet as herein above described, or obtained by the methods as herein above described.

Another object of the invention is the modified pig islet as herein above described, or obtained by the methods as herein above described, or the device of the invention for treating Type I Diabetes Mellitus or Type II Diabetes Mellitus in a subject in need thereof.

Another object of the invention is the modified pig islet as herein above described, or obtained by the methods as herein above described, or the device of the invention for regulating blood glucose levels in a subject in need thereof.

In one embodiment of the invention, the subject is a mammal, preferably a primate, more preferably a human.

### DEFINITIONS

In the present invention, the following terms have the following meanings:
“Isolated”: an organ, tissue or cell which have been separated from its natural environment. The term includes gross physical separation from the natural environment, such as, for example, removal from the donor animal, and alteration of the organ's, tissues, or cell's relationship with its neighboring cells or with which they are in direct contact by dissociation.
“Treating”: reducing or alleviating at least one adverse effect or symptom of a disease, disorder or condition associated with a deficiency in or absence of an organ, tissue or cell function.
“Administering”, “introducing” or “transplanting” (used interchangeably): placement of the organs, tissues, cells or compositions into a subject, for example a xenogeneic subject, by a method of route which results in the localization of the organs, tissues, cells or composition at a desired site.
“Recipient”: mammals, preferably humans, suffering from or predisposed to a disease, disorder or condition associated with a deficiency in or absence of an organ, tissue or cell function; “xenogeneic recipient”: a recipient into which cells of another species are introduced or are to be introduced.
“Disease, disorder or condition associated with a deficiency in or absence of an organ, tissue or cell function”: includes a disorder in which there is abnormal organ function. Such abnormal organ function includes an impairment or absence of a normal organ function or presence of an abnormal organ function.
“About”: preceding a figure means plus or less 10% of the value of said figure.
“Regulating blood glucose levels”: maintaining blood glucose levels within the parameters for a normal, non-diabetic individual of similar age and weight.
“Alginate”: salts of alginic acid. Alginic acid, which is isolated from seaweed, is a polyuronic acid made up of two uronic acids: D-mannuronic acid and L-guluronic acid. Alginic acid is substantially insoluble in water. It forms water-soluble salts with alkali metals, such as sodium, potassium, and, lithium; magnesium; ammonium; and the substituted ammonium cations derived from lower amines, such as methyl amine, ethanol amine, diethanol amine, and triethanol amine. The salts are soluble in aqueous media above pH 4, but are converted to alginic acid when the pH is lowered below about pH 4. A thermo-irreversible water-insoluble alginate gel is formed in the presence of gel-forming ions, e.g. calcium, barium, strontium, zinc, copper(+2), aluminum, and mixtures thereof, at appropriate concentrations. The alginate gels can be solubilized by soaking in a solution of soluble cations or chelating agents for the gel-forming ions, for example EDTA, citrate and the like.

### DETAILED DESCRIPTION

The present invention relates to an improved method for treating diabetic patients through the xenotransplantation of pig islets, wherein said pig islets are modified to enhance the production of insulin. According to the invention, one method for enhancing insulin production in pig islets consists in remodeling the structure (a/(cells ratio) of pig islets for enhancing glucagon production. Another method for enhancing insulin production in pig islets consists in transgenic modification of pig islets cells for enhancing glucagon production or for inducing production of an analog of glucagon. Without willing to be bound by a theory, the enhancement of the production of glucagon or analog thereof may induce an enhancement of cAMP concentration in cells, thus enhancing the production of insulin. By liaison on the beta cell membrane to the specific guanine nucleotide-binding protein (G-protein) -coupled receptor, glucagon or analog thereof will activate the enzyme adenylyl cyclase which will result in increased cAMP levels.

Cyclic AMP potentiates glucose stimulated insulin release through 2 main pathways. First, increased cytosolic concentrations of cAMP will activate the enzyme protein kinase A (PKA) which in turns will phosphorylate and activate proteins involved in the insulin secretory process (including PDX-1). Second, PKA-independent mechanism is involved in potentiated insulin secretion induced by cAMP. Binding of cAMP to Epac2 (exchange proteins directly activated by cAMP) is thought to cause conformal changes to G-protein Rap1 (Ras-related small GTPase 1) which will potentiates insulin exocytosis by enlarging the size of the pool of granules available for the direct release.

One object of the invention is a modified pig islet, preferably an isolated modified pig islet, capable of producing higher levels of glucagon than native pig islet or capable of producing a glucagon analog.

In one embodiment of the invention, said modified pig islet, preferably said isolated modified pig islet, is capable of secreting at least 1.1, 1.2, 1.3, 1.4, 1.5, 1.6, 1.7, 1.8, 1.9, two fold more, preferably at least 2.5 fold more, more preferably at least 3 fold more of glucagon than native pig islets in the conditions of Test A.

In one embodiment of the invention, said modified pig islet, preferably said isolated modified pig islet, is capable of secreting at least 1.2 fold more, preferably at least 1.3 fold more, more preferably at least 1.4 fold more, even more preferably at least 1.5 fold more, still more preferably at least 2 fold more of insulin than native pig islets in the conditions of Test A.

The method of Test A for determining secretion levels of glucagon or insulin is for example the following (see Examples):

1) overnight incubation of islets at 37° C., 5% CO₂/95% O₂ in RMPI medium containing 10% heat-inactivated FCS, 100 IU/ml penicillin, 100 μg/ml streptomycin, 5 mmol/l glucose,
2) 24 hr incubation of 100 of said islets in 12-wells plates in 2 ml RPMI at 5 mmol/l glucose, 15 mmol/l glucose or 15 mmol/l glucose+1 μmol/l Forskolin,
3) quantification of glucagon or insulin in recovered media by radioimmuno-assay.

In one embodiment of the invention, said modified pig islet, preferably said isolated modified pig islet, has a content of glucagon of at least two, three, four, fold more, preferably at least 5 fold more, more preferably at least 10 fold more, even more preferably at least 15 fold more, and even more preferably at least 20 fold more of glucagon than native pig islets in the conditions of Test B.

In one embodiment of the invention, said modified pig islet, preferably said isolated modified pig islet, has a content of insulin of at least 1.05, 1.1, 1.2 fold more, preferably at least 1.5 fold more, more preferably at least 2 fold more, even more preferably at least 2.5 fold more, still more preferably at least 3 fold more of insulin than native pig islets in the conditions of Test B.

The method of Test B for determining content levels of glucagon or insulin is for example the following (see Examples):
1) overnight incubation of islets at 37° C., 5% CO₂/95% O₂ in RMPI medium containing 10% heat-inactivated FCS, 100 IU/ml penicillin, 100 μg/ml streptomycin, 5 mmol/l glucose,
2) 24 hr incubation of 100 of said islets in 12-wells plates in 2 ml RPMI at 5 mmol/l glucose, 15 mmol/l glucose or 15 mmol/l glucose+1 μmol/l Forskolin,
3) transfer of islets in acid-ethanol for hormones extraction,
4) quantification of glucagon or insulin in recovered media by radioimmuno-assay.

According to an embodiment, the glucagon analog is selected from the group comprising glucagon like peptide 1 (herein after referred as GLP1 or GLP-1), PC (Protein Convertase) 1/3 (in alpha cells), TORC Bcl-2 (in beta cells).

In one embodiment, the production of the glucagon analog is assessed by methods well-known of the skilled artisan, such as, for example, bioassays (ELISA, RT-PCR, RT-qPCR, PCR, qPCR, Luminex assays . . . ) or immunoassays.

According to the invention, the islets are obtained from pigs.

In one embodiment of the invention, pig islets are obtained from young pigs aged of about 12 to 15 weeks.

In another embodiment of the invention, pig islets are obtained from pigs aged of less than about 6 months, preferably aged of less than about 2 months, more preferably aged of less than about 1 month.

In another embodiment of the invention, pig islets are obtained from adult pigs aged of more than about 2 years.

Young and adult pig pancreases have the advantage to provide a sufficient quantity of functional islets to perform xenotransplantation into primates. Moreover, they are able to respond to hyperglycemia within hours after transplantation.

In one embodiment of the invention, the pig donors are free of infectious microorganisms, in order to limit the risks of transmission of a disease during xenotransplantation. As an example, the islets may be extracted from AI pigs described in WO2006/110054, which is incorporated herein by reference. These pigs have been resident on the remote Auckland Island (New Zealand) and are free, or quasi-free, of porcine endogenous retrovirus (PERV) and other common pig infectious viruses including PCMV, PLHV, EMCV, HEV and PCV. Another example is specific pathogen-free (SPF) NZ Large White pigs raised under strict biosecurity.

According to one embodiment of the invention, the enhanced production of glucagon by the modified pig islets is obtained by remodeling the islet structure to increase the proportion of glucagon producing cells.

Another object of the invention is thus a modified pig islet, wherein the structure of said pig islet is modified to increase the proportion of glucagon producing cells.

Islets comprise beta cells capable of producing insulin and alpha cells capable of producing glucagon. Normal pig islets beta/alpha cells proportion is about 11.25/1 (beta cells/alpha cells).

According to said embodiment, the structure of the islet is remodeled by modifying the proportion of alpha and beta cells: the proportion of beta cells compared to alpha cells in the modified pig islets of the invention ranges from about 2.5/1 to about 5/1, preferably from about 2.5/1 to about 3.5/1, more preferably is about 2.5/1.

In one embodiment of the invention, the modified pig islets, preferably the isolated pig islets, of the invention exhibit the normal physiological structure of a pig islet, with alpha cells located in a ring at the periphery of the islet, while beta cells are located within the hub of the islet.

In one embodiment of the invention, the modified pig islets, preferably the isolated pig islets, have a size of less or equal to 250 μm, preferably of less or equal to 150 μM

Another object of the invention is a method for obtaining modified pig islets having a beta cells/alpha cells proportion ranging from about 2.5/1 to about 5/1, preferably from about 2.5/1 to about 3.5/1, more preferably is about 2.5/1, said method comprising administering Streptozotocin (STZ) to the pigs.

STZ is commercially available. In a preferred embodiment, STZ is provided by Sigma Aldrich (Bornem Belgium) under the reference 50130. Usually, STZ is solubilised in citrate buffer (25% Na citrate, 23% citric acid, 52% water, pH 4.5, percentage being in volume to the total volume of the solution) and filtered before use.

In one embodiment of the invention, pigs are treated with filter-sterilized STZ by injection in the external jugular vein.

In another embodiment of the invention, the dose of STZ injected ranges from 10 to 150 mg/kg of the pig body, preferably from 20 to 125 mg/kg, preferably from 30 to 100 mg/kg, more preferably from 30 to 50 mg/kg.

In another embodiment of the invention, the pigs are injected only once with STZ.

In another embodiment of the invention, modified pig islets as defined here above are isolated from the pancreas of STZ-treated pigs at least 2 months after STZ injection, at least 3 months, at least 6 months after STZ injection.

In another embodiment of the invention, the enhanced production of glucagon or the production of an analog thereof by the modified pig islets is obtained by transgenic modification of the pig islets cells.

Another object of the invention is thus a modified pig islet, preferably an isolated modified pig islet, wherein the production of higher levels of glucagon than native pig islet, or of glucagon analogs, is induced by transgenese.

Another object of the invention is thus a transgenic pig islet.

According to one embodiment, transgenic modification of pig islets cells induces the expression by said cells of genes selected from the group comprising Glucagon, Glucagon like peptide 1 (GLP1), PC (Protein Convertase) 1/3 (in alpha cells), TORC Bcl-2 (in beta cells). An example of porcine nucleic acid sequence for glucagon includes, but is not limited to, SEQ ID NO: 1. An example of porcine nucleic acid sequence for glucagon like peptide 1 includes, but is not limited to SEQ ID NO: 2. An example of cDNA encoding porcine GLP1 includes, but is not limited to, SEQ ID NO: 6.

An example of amino acid sequence of porcine GLP1 includes, but is not limited to SEQ ID NO: 7 (39 amino acids sequence): HDEFERHXEGTFTSDVSSYLEGQAAKEFIAWLVKGRGRR, wherein X is A or G.

Another example of amino acid sequence of porcine GLP1 includes, but is not limited to SEQ ID NO: 8 (amino acids 7-37 of SEQ ID NO: 7, corresponding to the active form of GLP-1, as described in Rowzee et al, 2011, Experimental Diabetes Research, 2011:601047): HXEGTFTSDVSSYLEGQAAKEFIAWLVKGRG, wherein X is A or G.

In one embodiment, the amino acid sequence of GLP-1, such as, for example, SEQ ID NO: 7 or SEQ ID NO: 8, may comprise a methionine (M) fused to its N-terminus part. An example of porcine nucleic acid sequence for PC 1/3 includes, but is not limited to SEQ ID NO: 3. An example of nucleic acid sequence for TORC Bcl-2 includes, but is not limited to SEQ ID NO: 4.

According to one embodiment, transgenic modification is carried out using viral vectors, preferably using viruses, more preferably using viruses selected from the group comprising Lentivirus, such as, for example, HIV vectors with different envelopes: VSV, gammaretroviral (MLV-A, RD114, GALV), Ross River Virus, Rabies, Measles; and Adeno-Associated-Vectors (AAV). In a preferred embodiment, the vector for transgenic modification is a Lentivirus.

According to one embodiment, the gene used for transgenic modification of the pig islets cells is under control of a tissue specific pig insulin promoter, with or without universal promoters such as UCOE promoters (resistant to silencing), CAGGS promoter (a combination of the cytomegalovirus (CMV) early enhancer element and chicken beta-actin promoter) or CMV (cytomegalovirus) promoter.

Another object of the invention is a vector comprising the sequence in nucleotides of glucagon, glucagon like peptide 1 (GLP1), PC (Protein Convertase) 1/3 or TORC Bcl-2 genes. Preferably, said vector is selected from the group comprising Lentivirus, such as, for example, HIV vectors with different envelopes: VSV, gammaretroviral (MLV-A, RD114, GALV), Ross River Virus, Rabies, Measles and Adeno-Associated-Vectors (AAV). Preferably, the sequence in nucleotides of glucagon, glucagon like peptide 1, PC (Protein Convertase) 1/3 or TORC Bcl-2 genes is under control of a tissue specific pig insulin promoter, such as, for example, insulin promoter. In one embodiment, said insulin promoter is specific of beta pig islet cells, leading to an expression of the transgene in beta cells only. An example of nucleic acid sequence of insulin promoter includes, but is not limited to, SEQ ID N: 9.

In one embodiment, the vector comprises the sequence in nucleotides of GLP1 and has the sequence SEQ ID NO: 5.

In one embodiment of the invention, the transgenic modification is carried out in vivo.

In one embodiment, the transgenic modification is carried out ex vivo, in order to generate transgenic pigs.

Methods for generating transgenic pigs using an ex-vivo method are well-known from the skilled artisan.

In one embodiment, the following method may be used for obtaining transgenic pigs:

An expression vector carrying a pig insulin promoter and the sequence of the transgene, preferably the sequence of GLP1 is developed. Primary Gal −/− and wild type fibroblasts are established from ear biopsy of pigs and cultured in vitro in DMEM/TCM199 with 10% FCS and 10 ng/ml of FGF in 5% CO2 and 5% O2.

Growing cultures are transfected. In one embodiment, transfection is carried out by electroporation. In one embodiment, transfection is carried out by chemical transfection.

In a preferred embodiment, transfection is carried out by a combination of smart electroporation and chemical transfection, such as, for example using Nucleofector (Amaxa).

Transfected cells are then expanded and frozen for nuclear transfer. An aliquot of said cells may be expanded to perform PCR analysis to determine the integration of the transgene, such as, for example, GLP1.

Oocytes are recovered from ovaries of slaughtered cycling female at the local slaughterhouse. Selected oocytes are matured in vitro in medium DMEM/F12 with 10% FCS in presence of gonadotropins for 42-44 h in 5% CO2 at 38.5° C. At the end of maturation, cumulus cells are removed and oocytes with the first polar body are selected for further processing.

In one embodiment of the invention, the methods used for nuclear transfer is based on the zona-free system. Zona pellucida is removed by pronase digestion with a short incubation time till zona pellucida starts to dissolve. Zona free oocytes are then stained with Hoechst and exposed to cytochalasine B before enucleation. Oocytes are layered in a row of microdrops individually and enucleated with a blunt micropipette.

In another embodiment of the invention, oocytes are prepared by conventional zona-enclosed method.

Cells used for nuclear transfer are grown to confluence and/or serum starved for 24-48 h to synchronise their cell cycle. Before manipulation they are trypsinised into single cell suspension and kept at room temperature until use.

For nuclear transfer, cells are spread at high dilution on a culture dish (drop of medium) just before use, enucleated oocytes are washed first in medium containing phytohemagglutinin and then immediately dropped over a cell and rolled over till there is strong contact between the two units (Vajta et al., 2003, Biology of reproduction, 68:571-8).

Subsequently the couplets (enucleated oocyte-somatic cell) are subjected to cell fusion. The couplets are transferred to an anionic media containing 0.3 M mannitol, 0.01 mM Mg, PVA and then to a fusion chamber. Fusion is obtained by delivering a double DC pulse of 1.2 Kv/cm for 30 μsec. Couplets that do not fuse are re-subjected to a second round of fusion. Fused couplets are activated within 1-2 h after fusion by double DC pulse of 1.2 KV/cm for 30 μsec in the fusion medium containing 1 mM Ca and incubated in 5 μM of cytochalasin B in mSOFaa medium for 3.5-4 h.

After activation the reconstructed zona free embryos are cultured in the modified ‘well of the well’ system (Vajta et al., 2000, Molecular Reproduction and Development, 55:256-64) in microdrops under mineral oil to prevent adhesion between embryos.

For in vitro culture 20 μl microdrops of mSOFaa (Galli et al., 2003, Cloning Stem Cells, 5: 223-232) under oil are prepared and then 10 to 15 small depressions are made using a blunt small metal device. In each depression one embryo is accommodated for all the culture period. On day 3 of culture half of the medium is replaced with fresh media. On day 5 embryo development is evaluated. Compacted morula and early blastocysts are transferred to the uterus of synchronised recipients.

Pregnancies is diagnosed by ultrasound on day 25 of gestation. Recovered fetuses or newborn animals are subjected to analysis to determine transgene expression in the islets. The pancreases of the fetuses are analysed as well as storing in liquid noitrogen a cells for future cloning. Based on the immunicytochemestry findings the best expressing fetuses are subjected to re-cloning to generate the animals required for the islets isolation. In this case all the pregnancies are allowed to go to term to generate live animals.

In another embodiment of the invention, the transgenic modification is carried out in vitro. Advantageously, an ex vivo gene transfer approach is carried out. According to one embodiment, transfection of pig islet cells is carried out according to procedures well known in the art such as lipofectamine or polyethylenimine transfection or electroporation.

Consequently, another object of the invention is a transgenic pig islet cell, preferably an isolated transgenic pig islet cell, wherein the expression of a gene, selected from the group comprising Glucagon, glucagon like peptide 1, PC (Protein Convertase) 1/3 (in alpha cells) and TORC Bcl-2 (in beta cells), has been induced. Another object of the invention is a transgenic pig islet cell, wherein the expression of Glucagon has been enhanced. Another object of the invention is a transgenic pig islet cell, wherein the expression of GLP-1 has been induced.

Another object of the invention is a transgenic pig islet, preferably an isolated transgenic pig islet, wherein the expression of a gene, selected from the group comprising Glucagon, glucagon like peptide 1, PC (Protein Convertase) 1/3 (in alpha cells) and TORC Bcl-2 (in beta cells), has been induced.

In one embodiment of the invention, the transgenic pig islets, preferably the isolated transgenic pig islets, of the invention exhibit the normal physiological structure of a pig islet, with alpha cells located in a ring at the periphery of the islet, while beta cells are located within the hub of the islet.

In one embodiment of the invention, the transgenic pig islets, preferably the isolated transgenic pig islets, have a size of less or equal to 250 μm, preferably of less or equal to 150 μm.

Another object of the invention is thus a transgenic pig, wherein the expression of a gene, selected from the group comprising Glucagon, glucagon like peptide 1, PC (Protein Convertase) 1/3 (in alpha cells) and TORC Bcl-2 (in beta cells), has been induced.

The present invention thus also relates to a method for obtaining a transgenic pig islet cell, a transgenic pig islet or a transgenic pig according to the invention.

In another embodiment of the invention, transgenic pig islets as defined here above are isolated from the pancreas of transgenic pigs aged of less than 6 months, preferably aged of less than 3 months, more preferably aged of less than 2 months.

Methods for the isolation of pig islets (from wild-type, transgenic or STZ-treated pigs) are well known for the one skilled in the art.

In one embodiment, the isolation of pig islets is carried out according to the protocol described in Dufrane et al., Xenotransplantation, 2006 and Dufrane et al., Transplantation, 2006.

According to one embodiment, the isolation protocol comprises a step of exsanguinations of pigs, in order to reduce the pancreatic blood content. Briefly, after cerebral death, animals are kept with the heart beating until the time of evisceration. Blood exsanguination is performed by incision of the carotid artery and jugular vein, and the animals are suspended for 1 to 10 minutes, preferably for 4 to 7 minutes by the back legs.

Briefly, pancreases are dissected ex vivo. According to one embodiment, the dissection of pancreases is performed with a warm ischaemia ranging from 5 to 25 minutes. The pancreatic duct is then evidenced and cannulated with an 18-gauge catheter. The gland is then distended with cold storage solution by means of a perfusion solution. According to an embodiment, 1 mL of perfusion solution is used per gram of tissue. Pancreases are stored submerged in preservation solutions. According to one embodiment, the preservation solution is classic University Wisconsin (UW, n=6) or in modified-UW (UW-M; no hydroxyethyl starch and low K⁺/high NA⁺).

Extracted pancreases are then digested. According to a preferred embodiment, pancreas dissociation is performed with Liberase DL Research Grade (Dispase Low) enzyme, preferably provided by Roche/Boehringer Mannheim. The enzyme is dissolved at cold temperature, preferably at a temperature ranging from 4 to 12° C., preferably at about 8° C., in UW-M solution at a concentration ranging from 0.1 to 1 mg/mL, preferably at a concentration of about 0.5 mg/mL.

According to one embodiment, the pancreases are digested using the dynamic method, as described by Ricordi et al., 1986. Briefly, pig pancreases distended with the enzyme are sliced, loaded on a Ricordi chamber (preferably made of 316 1 stainless steel with seven glass marbles) and digested at 37° C. with a heating circuit, and the chamber is agitated manually. When a significant number of isolated islets appeared in the samples, the digestion circuit is cooled by addition of cold Ham-F10 medium containing 10% NCS in order to reduce enzyme activity. Cold medium is then perfused for 25 to 40 min. Islets, cells and debris are collected in 250 mL tubes and centrifuged at 4° C. (630 g for 3 min). All cellular pellets are pooled and suspended in 200 mL Ham-F10 medium.

According to another embodiment, the pancreases are digested using the static method, as described by O'Neil et al., 2001. Briefly, the pancreas is infused with a two to four fold volume (mL/g) of liberase PI. The pancreas is injected in order to achieve an adequate distension, placed in a sterile 1 L Nalgene jar and digested by static incubation at 37° C. for 45 to 60 minutes. Digestion is terminated by the addition of Ham-F10+20% NCS based on the visual inspection of the gland. The cell suspension is filtered through a stainless steel mesh with a pore size of 1000 μm and diluted in Ham-F10+20% NCS. Digested tissue is then passed over a bed of 6 mm glass beads and through a stainless-steel mesh screen. The tissue effluent is collected with 3 to 4 L of cold Ham-F10+10% NCS in 250 mL conical tubes and centrifuged at 700 rpm at 4° C. Islets, cells and debris are collected in 250 mL tubes and centrifuged at 4° C. (630 g for 3 min). All cellular pellets are pooled and suspended in 200 mL Ham-F10 medium.

According to one embodiment, following their isolation, the pig islets are purified. According to one embodiment, the purification of the pig islets is carried out using a discontinuous Ficoll gradient as described in Dufrane et al., Xenotransplantation, 2006. Briefly, isolated islets are purified at 4° C. using a discontinuous Ficoll gradient, preferably a FIcoll Euro-Collins gradient. The post-digestion cellular pellet, suspended in 75 mL of Ficoll Euro-Collins solution (density=1.1 g/cm³) is placed in a flat-bottomed tube. Lower gradients of Ficoll are then added sequentially (50 mL of 1.096 g/cm³, 50 mL of 1.060 g/cm³ and 20 mL of Ham-F10 medium). Ham-F10 medium is F-10 nutrient mixture medium, and is commercially available, for example it is provided by N.V. Invitrogen, Belgium. After centrifugation of the gradient tubes at 856 g for 17 minutes, islets are collected from 1.1/1.096 and 1.096/1.060 interfaces. Islets from each interface are suspended in two tubes containing 50 mL Ham-F10+10% NCS serum (NCS is for newborn calf serum, and is commercially available, for example it is provided by Biochrom AG, Germany). The tubes are centrifuged at 280 g for 3 minutes, the supernatant is removed, and the cells are washed with 150 mL Ham-F10 medium. This procedure is repeated three times and, finally, the islets are suspended in 200 mL Ham-F10 medium.

Another object of the invention is a device comprising the modified pig islet, preferably the isolated modified pig islet of the invention. In one embodiment, the device of the invention comprises a modified pig islet, preferably an isolated modified pig islet, as described herein above, preferably a modified pig islet having a beta cells/alpha cells proportion ranging from about 2.5/1 to about 5/1, preferably from about 2.5/1 to about 3.5/1, more preferably is about 2.5/1. In another embodiment, the device of the invention comprises a transgenic pig islet, preferably an isolated transgenic pig islet as described here above. In another embodiment, the device of the invention comprises the composition as described here above.

According to one embodiment, the device of the invention is an implantable or transplantable device.

According to another embodiment, the device of the invention is an injectable device.

According to another embodiment, the device of the invention is biodurable, which means that it shows an improved biostability when implanted or injected to a subject. This improved biostability enables the cells present in the device to remain within a living body for a longer period than is currently the case, which will result in improved treatment efficacy.

In one embodiment of the invention, the device may be a vascularized subcutaneous collagen tube, as described in WO02/32437, in order to allow the development of a prevascularized autologous collagen reservoir for the placement of the islet. In brief, a closed ended tube of stainless steel mesh containing a loosely fitting Teflon rod is inserted subcutaneously in the intended graft recipient. Six weeks later the rod is removed, leaving a highly vascularized tube of collagen. The islets are inserted into the vascular tube which is then sealed with a Teflon stopper.

In another embodiment of the invention, the device may be a matrix preparation including preparation of gelatin, collagen, and natural carbohydrate polymers.

In another embodiment of the invention, the device may be a plasma thrombin clot—autologous plasma clots produced with allogeneic thrombin.

In another embodiment of the invention, the device may be a suitable biocompatible material as a capsule to provide additional immune protection of the transplanted islets. Encapsulation systems are well-known in the art. Advantageously, the capsule is made of a semi-permeable membrane, which is permeable to glucose, nutrients and insulin, but not to humoral/cellular immune components.

In one embodiment, the semi-permeable membrane is made of a material selected from the group comprising alginate, nitrocellulose, acrylonitrile, agarose and polytetrafluoroethylene. In a preferred embodiment, the semi-permeable membrane is made of alginate.

In another embodiment, the device may be an encapsulation system for living cells, as described in WO2007/046719. According to this embodiment, the encapsulation system comprises a biodurable composition comprising alginate which is high in mannuronic acid specifically containing between about 50% to 95% mannuronic acid residues, and a polycation having a polydispersity index of <1.5, such as poly-L-ornithine. The encapsulation system may be a biocompatible microcapsule prepared using the composition hereabove described, and comprising a core layer of high mannuronic acid alginate cross-linked with a cationic cross-linking agent, an intermediate layer of polycations having a polydispersity index of less than about 1.5 forming a semi-permeable membrane, and an outer layer of high mannuronic acid alginate, the microcapsules comprising living cells within the core layer.

In another embodiment, the device may be a microcapsule as described in WO02/032437: sodium alginate used for this procedure is extracted from raw material sources (seaweed) and prepared in a powdered ultrapure form. The encapsulation procedure involves extruding a mixture of islets and sodium alginate solution (1.6%) through a droplet generating needle into a bath of gelling cations (calcium chloride). The islets entrapped in the calcium-alginate gel are then coated with positively charged poly-L-ornithine followed by an outer coat of alginate (0.05%). The central core of alginate is then liquefied by the addition of sodium citrate. Most capsules contain 3 islet cells and have a diameter of 300 to 400 μm.

In another embodiment, the device may be a macrocapsule as described in WO2010/032242. WO2010/032242 discloses a system for transplanting and immunoisolating cells (e.g., functional cells, typically, islets of Langerhans) by an artificial membrane provided by macroencapsulation of the cells in a hydrogel such as an alginate matrix. The hydrogel macroencapsulating the islets is formed so as to have a planar, geometric configuration, e.g., a slab, a sheet, or a disc. Typically, the alginate structure has at least one substantially flat surface. The alginate comprises an ultrapure grade alginate and a defined composition that is cross-linked so as to encapsulate the cells or tissue segments in a hydrogel. Typically, the alginate slab houses islets at a density of 2,000-8,000 islets/cm². The alginate macroencapsulating the islets typically has a concentration of guluronic acid of less than 50% such that the slab is flexible enough to conform to the shape of the kidney and fit within the subcapsular space thereof, but strong enough to maintain its overall physical characteristics. Additionally, the alginate comprises a dry matter content that is greater than 1.5% such that the slab is strong and stable enough to withstand forces. Typically, the macroencapsulated islets slab provides a ratio of volume of islets to volume of alginate of at least 1:10 (i.e., 10% islets by volume). For some applications, the alginate used to encapsulate the islets is supplemented with collagen. In some applications, the islets are disposed in the center of a primary alginate slab, and a supplementary alginate layer surrounds the encapsulated islets within the primary alginate slab. In such an application, a layer of medical grade collagen may be used in combination with the supplementary alginate layer.

In another embodiment of the invention, the device may be a cellular device as described in WO2007/144389, said device comprising (a) a collagen matrix having a first side and a second side; (b) a first cell layer absorbed onto the first side of the collagen matrix; and (c) a first gelled alginate layer and a second gelled alginate layer; wherein the first gelled alginate layer completely covers the first side of the collagen matrix and the first cell layer; and wherein the second gelled alginate layer completely covers the second side of the collagen matrix.

According to an embodiment, freshly isolated pig islets are encapsulated in an SLM 100 alginate matrix (FMC BioPolymer, Norway) with the Inotech Encapsulation AG Device (Dottikon, Switzerland).

Preferably, the device of the invention is sterilized before implantation or injection into a patient body. Advantageously, the sterilization comprises γ-irradiation, E-beam, ethylene oxide, autoclaving or contacting the device with alcohol prior to addition of the liquid component or contacting with NOx gases, hydrogen gas plasma sterilization.

Preferably, the device possesses a low content of endotoxins. In some embodiments, the cellular device possesses an endotoxin level of less than 100 endotoxin units (EU)/g, less than 90 EU/g, less than 80 EU/g, less than 70 EU/g, less than 60 EU/g, less than 50 EU/g, less than 40 EU/g, less than 30 EU/g, less than 20 EU/g, less than 10 EU/g, less than 5 EU/g, or less than 1 EU/g.

In another embodiment of the invention, the device may be an encapsulation chamber as described in WO02/060409, said device comprising cells, such as, for example islet cells, producing a biologically active substance, such as, for example, insulin, and comprising at least one semi-permeable membrane. The semi-permeable membrane of said device may comprise a biocompatible porous polycarbonate film, wherein the porous polycarbonate film is modified on surface by the creation of polar sites, and wherein the porous polycarbonate film is coated by at least one hydrophilic polymer, such as, for example, cellulose, polyacrylamide, polyvinylpyrrolidone, copolymer of vinyl acetate, polyethylene glycol, hydrophilic poly(meth)acrylate, polyoside and chitosan.

Another object of the invention is a method for treating Type I Diabetes Mellitus or Type II Diabetes Mellitus in subjects in need thereof, comprising the administration of modified pig islets of the invention or devices of the invention.

Another object of the invention is a method for regulating blood glucose levels in subjects in need thereof, comprising the administration of modified pig islets of the invention or of devices of the invention.

Another object of the invention is modified pig islets or devices of the invention for treating or for use in treating Type I Diabetes Mellitus or Type II Diabetes Mellitus in subjects in need thereof.

Another object of the invention is modified pig islets or devices of the invention for regulating blood glucose levels in subjects in need thereof.

According to an embodiment, the subject to whom the treatment is administered suffered of Type I Diabetes Mellitus or of Type II Diabetes Mellitus. Preferably, the subject or patient is a mammal, preferably a primate, more preferably a human.

In an embodiment, the method of treatment of the invention comprises the administration, through implantation, transplantation or injection, of modified pig islets cells or devices of the invention. Preferably, the administration is made subcutaneously, intraperitoneally, intramuscularly, in or under the kidney capsules.

According to one embodiment, the number of pig islets cells administered ranges from 10000 to 50000 IEQ/kg of body weight, preferably from 30000 to 50000 IEQ/kg of body weight. IEQ means pig islets equivalents.

According to an embodiment, when devices are used, one or more devices may be administered.

In one embodiment, the method of treatment of the invention may also further comprise the administration of an immunosuppressive treatment. Advantageously, the immunosuppressive treatment comprises or consists of the administration of at least one product selected from the group comprising daclizumab, tacrolimus, rapamycin, mycophenolate mofetil, cyclosporine, deoxyspergualin or deoxyspergualin analogue, soluble complement receptor 1, anti-CD154 antibody, ATG, methylprednisolone, anti-IL-2R antibody, basiliximab, FTY720, everolimus, leflunomide, sirolimus, belatacept, CTLA4-Ig, cobra venom.

Preferably, when modified pig islets are administered without device, an immunosuppressive treatment is carried out.

### BRIEF DESCRIPTION OF THE DRAWINGS

FIG. 1: the alpha cells proportion and content before isolation. (A) The proportion of alpha cells increased with the dose of STZ (r²=0.88; p<0.05). Native islets presented a lower alpha cell proportion than other groups (*: p<0.001). Pigs treated with 30 mg/kg STZ showed lower alpha cell proportion than 75, 100 mg/kg and human (**: p<0.001). Islets from pigs treated with 50 mg/kg STZ presented lower alpha cell proportion than 75 and 100 mg/kg STZ (§: p<0.001). Treatment of pigs with 50 mg/kg STZ is the only dose allowing similar alpha/beta cell proportion than in humans.

By using the correlation obtained between the dose and the proportion of alpha cells, a theoretical increase of 30% of the proportion of alpha cells would be obtained with a dose of 50 mg/kg STZ. (B) The number of alpha cells per islet was lower in native islets in comparison to other groups (*: p<0.001). Pigs treated with doses of 30 and 50 mg/kg presented a lower number of alpha cells per islet than pigs treated with 75 mg/kg STZ and humans (§: p<0.001).

FIG. 2: the pancreatic hormonal content. (A) A correlation was observed between the dose of STZ and the pancreatic insulin content (r²=0.77; p<0.05). By extrapolation of this correlation, it appears that a theoretical reduction of 30% in the pancreatic hormonal content would be obtained with a dose of 30 mg/kg STZ.

Native pigs and humans demonstrated higher pancreatic insulin content than pigs treated with 75 and 100 mg/kg STZ (*: p<0.01). A dose of 30 mg/kg STZ showed higher insulin content than treatment with 100 mg/kg (§: p<0.05). (B) Pancreatic glucagon content in function of the dose of STZ. Note higher glucagon content in human pancreas than in native islets as well as in pancreas of pigs treated with 30, 50 and 75 mg/kg STZ.

FIG. 3: alpha cells proportion and content after isolation. (A) Native islets showed lower alpha cells proportion than STZ-treated islets and humans (*: p<0.05). Pigs treated with 30 mg/kg STZ presented islets of 150-200 μm presenting a lower alpha cell proportion than 50 mg/kg group (§: p<0.001) but also a higher alpha cell proportion than humans (**: p<0.005). (B) Native islets presented a lower number of alpha cells than treated islets and humans (*: p<0.05). Islets from STZ treated pigs (30 and 50 mg/kg) demonstrated a higher number of alpha cells per islets than humans (**: p<0.001). Islets from pigs treated with 30 mg/kg showed a lower number of alpha cells than 50 mg/kg (§: p<0.001).

FIG. 4: Islet glucagon content and release after glucose stimulation. Islets from pigs treated with 50 mg/kg STZ demonstrated higher glucagon content and release after G5 and G15 stimulation than native and 30 mg/kg treated islets (*: p<0.01; §: p<0.05).

FIG. 5: Islet insulin content and release after glucose stimulation. Islets from pigs treated with 30 mg/kg STZ demonstrated higher insulin secretion after G5 and G15 stimulations (p<0.005). Islets from pigs treated with 30 mg/kg STZ also demonstrated higher insulin content after G5 stimulation (*: p<0.05).

### EXAMPLES

The present invention is further illustrated by the following examples.

### Example 1

### Material and Methods

### Human and Pig Pancreatic Donors Source:

Twelve human pancreases were obtained from cerebral death donors obtained from multiorgan donors through the Eurotransplant Network (Leiden, the Netherlands), according to an ethical committee (protocol UCL-HIA-001, authorization 2001/79) in accordance with the principles of the Declaration of Helsinki of 2000 and the guidelines defined by the Belgian authorities. The donors were aged 28-62 years. Thirty-two pancreases of young Landrace pigs (12-15 weeks old, weighing 42.2 to 50 kg) were harvested (Rattlerow Seghers [Lokeren, Belgium]).

### Pig Pancreatic Tissue Remodelling:

### Streptozotocin Injection

Five doses (0, 30, 50, 75, 100 mg/kg) of filter-sterilized Streptozotocin (STZ) (Sigma, Bornem, Belgium) were tested. STZ was solubilised in citrate buffer (25% v/v Na citrate, 23% v/v citric acid, 52% v/v H₂O, pH 4.5) and filtered before injection in the external jugular vein (Dufrane, Transplantation, 2006).

### In Vivo Metabolic Function

Intravenous glucose tolerance tests (IVGTTs) were performed prior and 3 months after STZ injection under general anesthesia (induction by Zoletil (VIRBAC, Carros, France) IM at a dose of 6 mg/kg, maintained by intubation and inhalation of Enflurane (0-1.5%), nitrous oxide and oxygen. A catheter was introduced in the extern jugular vein and a solution of 0.5 g glucose/kg was injected. Blood samples were taken prior and 1, 3, 5, 10, 20, 30, 60, 90 minutes after complete injection of glucose. Glycaemia was measured on these samples (AccuChek, Roche, Brussels, Belgium) and serum was conserved for radioimmuno assay (RIA) quantifications (insulin, c-peptide).

### Pancreatic Tissue Processing:

### Procurement of Pancreas

Human abdominal organs were perfused in situ with University of Wisconsin (UW) solution (4° C.). The warm ischemia time varied between 12 and 40 minutes. After removal, the pancreases were shipped in cold UW solution from the donor centre to the isolation laboratory. The cold ischemia time varied from 2 to 10 hours (Dufrane et al, Pancreas 2005).

Young pigs underwent anesthesia (see above). The pigs were subjected to laparotomy, the infrarenal aorta and vena cava (IVC) were clamped, and the thoracic aorta was isolated and clamped, and then in situ intraaortic perfusion was carried out with a 4° C. physiologic solution. During the in situ cold perfusion, the entire pancreas was completely dissected and freed. The pancreatic duct was revealed and an 18-gauge catheter was inserted into the pancreatic duct. The splenic and pancreaticoduodenal vessels were clamped, and the whole pancreas was harvested (except the ventral branch). The pancreas was then weighed and placed on ice to avoid any warm ischemia.

### Pancreas Structure Prior to Isolation

### Immunohistochemistry:

The cellular composition of islets was determined by immunohistochemistry in small samples of human and pig pancreas taken during dissection of the gland. After overnight fixation in formol at room temperature, the small biopsies were embedded in paraffin and cut in 5 μm sections. These sections were thereafter deparaffinized, rehydrated and washed with a solution of Tris-HCl-Buffered solution 0.05 mol/L (TBS, pH 7.4). After inactivation of endogenous peroxidase by a 30-minute incubation in 0.3% H₂O₂, the sections were incubated with 10% normal goat serum (NGS) in TBS for 30 minutes. The sections were then incubated overnight at 4° C. with glucagon rabbit polyclonal antibody (BD Bioscience, Erembodegem, Belgium) diluted at 1:100. After washes in TBS, the slides were incubated for 30 minutes with anti-rabbit-IgG (1:500) and these antibodies detected by EnVision anti-rabbit system (Dako A/S, Glostrup, Denmark) for 1 hour at room temperature. The peroxidase activity was revealed by immersion of sections for 10 minutes in a solution 3,3′-diaminobenzidine hypochloride (3,3′-diaminobenzidine, 50 mg/100 mL at pH 7.4; Fluka, Buchs, Switzerland), supplemented with 0.01% hydrogen peroxide. The glucagon staining was followed by the detection of the β-cells by alkaline phosphatase anti-alkaline phosphatase system (APAAP). After washing, aspecific sites were again inhibited by a 30 minutes incubation with 10% normal goat serum (NGS) in TBS. The slides were thereafter incubated overnight with mouse insulin monoclonal antibody (Abcam, Cambridge, UK) diluted at 1:800. After washes in TBS, the slides were incubated for 30 minutes with anti-mouse-IgG-biotin conjugate and then with streptavidin-peroxydase conjugate at a dilution of 1:500 (Roche Diagnostics, Mannheim, Germany), and the reaction was revealed by the kit fast red/naphtol AS MX tablet sets (Sigma, St. Louis, Mo.).

After washes, the slides were counterstained with Mayer's hemalun and mounted with a hydrosoluble mounting (Dako, Carpenteria, Calif.).

### Histomorphometry Analysis:

Islets from human and pig pancreas were compared for cellular proportion and geometric distribution of α and β cells (centre/periphery). Following the islets size, 3 groups of 5 islets (50-99, 100-149 and 150-200 μm) were compared in native/modified young pig versus human pancreas.

The number of small β-cell clusters (≦3 insulin-immunoreactive cells) in the total area of the slide was also assessed. The number of these clusters was manually counted through the entire slide. The area of the slide was measured with ImageJ 1.43i software after capture of pictures of slides with a camera Nikon Coolscan 5000, accessory FH-G1) and calibrated with a stage micrometer to correspond to the number of pixels evidenced on the image analyser.

### Hormonal Content for Insulin/Glucagon:

For pancreatic hormonal content, insulin and glucagon were first extracted from small biopsies of pancreas taken in the 3 zones (head, body, tail) before being quantified by radio immuno assay (RIA).

For the extraction, ˜1 g of pancreas was cut into small fragments (˜1-2 mm³) and 5 mL of a solution composed of 75.75% CH₃CH₂OH, 24.22% H₂O, 0.03% HCl were added. The following steps were performed with the samples kept on ice. Samples were mixed (Ultra-Turrax T25 [Janke & Kunkel IKA—Labortechnik, Staufen, Germany]) for 20 seconds 4 times and 5 mL of a solution of 75% CH₃CH₂OH, 22% H₂O, 3% HCl were added to the mixture. The samples were then sonicated 4×20 seconds (Sonifier B 12 [Branson Sonic Power Company, Danbury, Conn., USA]) and centrifugated 10 minutes at 4° C. at 1500 rpm. The first supernatant formed was removed and kept at −20° C. 2 mL of a solution of 75% CH₃CH₂OH, 23.5% H₂O, 1.5% HCl were added to the precipitate and a second step of sonication was performed. The samples were then kept at −20° C. overnight. After a step of centrifugation 10 minutes at 4° C. at 1500 rpm, the second supernatant was removed and kept at −20° C. Thereafter, 2 mL of a solution of 75% CH₃CH₂OH, 23.5% H₂O, 1.5% HCl were added to the precipitate and the mixture was sonicated and centrifuged 10 minutes at 4° C. at 1500 rpm and the third supernatant removed. The three supernatants were then pooled and constituted the pancreas extract. Human insulin specific RIA KIT and Glucagon RIA KIT (Millipore, Billerica, Mass., USA) were used for the determination of the hormonal content in human/pig pancreatic extractions as well as in isolated islets (see below). The content of insulin and glucagon in the extracts was performed following the instructions of the manufacturer. Briefly, 100 μL of hydrated ¹²⁵-I-Insulin or ¹²⁵-I-Glucagon and 100 μL of specific antibodies were added to 100 μL of pancreas extract diluted at 1:500, 1:1000, 1:3000 and 1:9000; 100 μL of pure sera; 100 μL of media (1:20) or 100 μL of modified islet extract (1:200). After incubation overnight at room temperature, 1 mL of precipitating reagent were added, followed by incubation 20 minutes at 4° C. The samples were then centrifuged 20 minutes at 2000-3000×g at 4° C. The supernatants were immediately removed and the contents of insulin and glucagon were determined by a Wallac Wizard 1470 Automatic gamma counter (GMI Inc, Ramsey, Minn., USA) for 1 minute.

Islet Structure after Isolation

### Islet Isolation

(i) Human islets were isolated using the simplified method previously described (Dufrane, Pancreas, 2005). Briefly, after being distended with enzyme (Liberase HI (Roche/Boehringer Mannheim, Brussels, Belgium; 0.5 mg/mL) dissolved in modified UW solution), the pancreas was cut into +/−3×4 cm pieces and digested by a dynamic incubation at 37° C. The pieces were placed in a sterile 1 L Nalgene jar which was closed and agitated by hand for ˜45 minutes. The digest was then filtered in a 500 μm filter and purified in a ficoll grade (densities 1.096, 1.080, 1.070 and 1.060) (Mediatech Cellgro). The islets were hand-picked, washed and suspended in Ham-F10+10% Human Serum (Cambrex).

(ii) Young pig islets treated with selected doses of STZ (0, 30, 50 mg/kg) were isolated following a method previously described by Dufrane et al, Xenotransplantation, 2006. Briefly, after being completely dissected, the pancreas were digested by a modified static digestion method: infusion of the pancreas with Liberase DL Research Grade (Roche/Boehringer Mannheim, Brussels, Belgium; 0.43 mg/mL) dissolved in modified UW solution; placement in a sterile 1 L Nalgene jar for digestion by static incubation at 37° C. for 30-50 minutes; filtration in a 500 μm filter; purification in a ficoll grade (densities 1.090, 1.060 and 1.010) (Mediatech Cellgro); washing and suspension of the islets in Ham-F10 (Gibco)+10% New Born Calf Serum (NCS; Merck-Eurolab, Overijse, Belgium).

### Histology

The cellular composition of human and pig isolated islets was determined by immunohistochemistry. After overnight fixation in formol at room temperature, aliquots of islet preparations were embedded in paraffin and cut in 5 μm sections. Insulin and glucagon staining were performed as described above.

Alpha and β cells proportion and localization (for pig and human) inside islets were compared in isolated islets from 0, 30 and 50 mg/kg STZ pigs in 3 groups of 5 islets (50-99, 100-149 and 150-200 μm).

### In Vitro Islet Functionality

After overnight incubation at 37° C., 5% CO₂/95% O₂ in RMPI medium containing 10% heat-inactivated FCS, 100 IU/ml penicillin, 100 μg/ml streptomycin, 5 mmol/l glucose, function of islets was assessed by 24 hr incubation of 100 islets in 2 ml RPMI at 5 (G5), 15 (G15) mmol/l glucose and 15 mmol/l glucose+1 μmol/l Forskolin (Fsk) (Calbiochem-Behring, San Diego, Calif.) (added from a mmol/l stock solution in DMSO). Three replicates per concentration were performed. Media were thereafter recovered for insulin and glucagon quantification and islets were transferred in acid-ethanol for hormones extraction and quantification by radioimmuno-assay (see above).

### Statistical Analysis

Values are presented as means±SD (except when otherwise specified). The one-sample Kolmogorove Smirnov test was used to assess the normal distribution of values. The statistical significance of differences between experimental groups was tested by one-way analysis of variance (ANOVA) with a Bonferroni's post hoc test. The statistical tests were carried out with Systat version 8.0. Differences were considered to be significant at p<0.05.

### Results

The doses of 0, 30, 50, 75 and 100 mg/kg STZ used to modify the structure of pig islets are not sufficient to induce dysfunction in glucose metabolism in pigs since IVGTT curves are close to those observed in control animals. In contrast, pancreatectomized pigs and pigs treated with 150 mg/kg STZ showed significant higher AUC for IVGTT curves with a slower decreasing glucose phase. Therefore, a dose of 30, 50, 75 and 100 mg/kg STZ, but not of 150 mg/kg, may be used for pig islets remodeling.

### Pancreas Structure Prior to Isolation

### Histomorphometry Analysis:

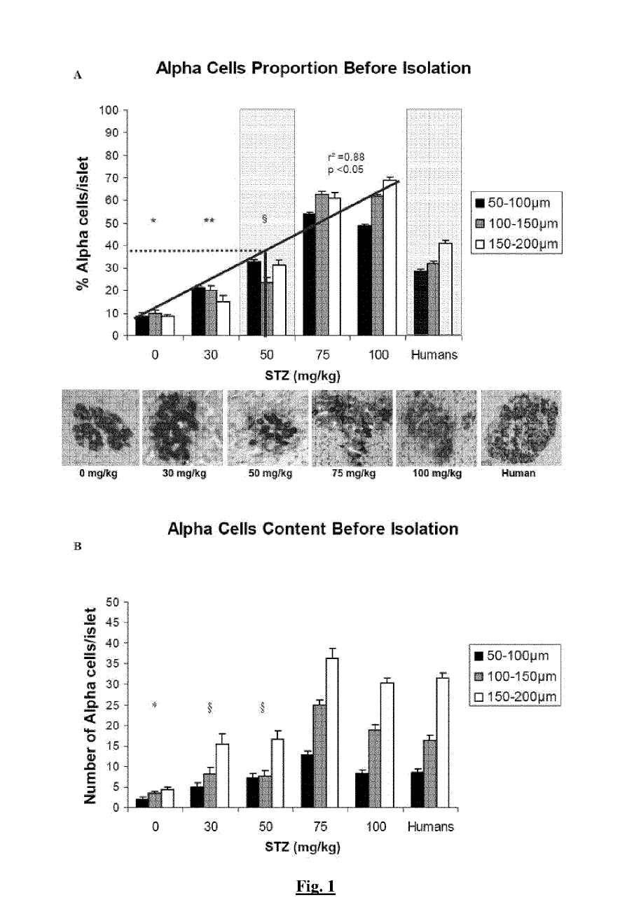

After quantification by histomorphometry of the proportion of α and β cells per islet prior to isolation, it appears that native pig pancreatic islets of each size (50-200 μm) showed a lower alpha-cell content than human islets (p<0.001) (FIG. 1A). Native pig islets also presented a significant lower number of alpha and beta cells per islet in comparison to human islets of each size (p<0.05) (FIG. 1B).

Doses of 75-100 mg/kg STZ were excessive since they induced a higher proportion of alpha cells (p<0.001), and concomitantly, a lower proportion of beta cells in each islet size in comparison to humans (FIG. 1A). Pigs treated with doses of 75 mg/kg STZ presented a higher number of alpha cells (p<0.05) and a lower number of beta cell per islet of 50-100 μm than humans (p<0.05). A dose of 100 mg/kg STZ also induced a lower number of beta cells per islet of 50-100 μm in comparison to humans (p<0.001) (FIG. 1B).

A linear correlation (r²=0.88; p<0.05) was observed between the STZ dose and a/f3 cell proportion (FIG. 1A). By linear regression, a theoretical reduction of 30% of the proportion of β cells, and concomitantly, an increase of 30% in the proportion of α cells (similar to humans) would be obtained with a dose of 50 mg/kg STZ (FIG. 1A). Moreover, the number of a (FIG. 1B) and β cells per islet increased significantly from doses of 30 mg/kg STZ.

Pigs treated with doses of 30-50 mg/kg STZ demonstrated a similar alpha/beta cells proportion to humans. However, in large islets (150-200 μm), 30-50 mg/kg STZ induced a lower alpha cell proportion in comparison to human islets (p<0.05) (FIG. 1A).

A lower number of alpha cells per islet was observed for islets of 50-100 μm and 150-200 μm from pigs treated with 30-50 mg/kg STZ than humans (p<0.001) (FIG. 1A).

Pigs treated with 50 mg/kg STZ showed a similar number of alpha cells per islet of 100-150 μm to humans (FIG. 1B).

Human islets of 50-100 μm demonstrated a higher number of beta cells per islet than pigs treated with 30 mg/kg (p<0.05) and a similar number of beta cells to 50 mg/kg STZ islets. Human islets of 100-150 μm presented a higher number of beta cells than pigs treated with 50 mg/kg (p<0.05) and a similar number of beta cells to pigs treated with 30 mg/kg.

No sign of beta cell regeneration (from duct or exocrine tissue) was found.

### Hormonal Content for Insulin/Glucagon:

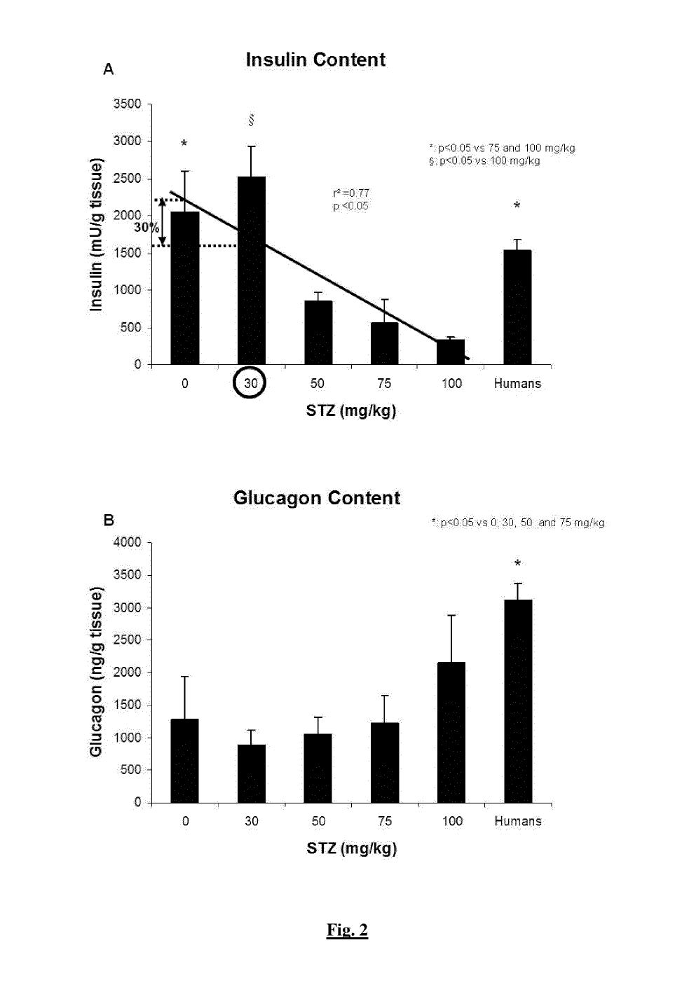

Pancreatic insulin content was similar in native pigs (0 mg/kg STZ) and in humans (FIG. 2 A).

Doses of 75-100 mg/kg STZ induced a significant reduction of the pancreatic insulin content (p<0.05) (FIG. 2 A) inadequate for tissue remodeling.

Pigs treated with doses of 30-50 mg/kg STZ presented similar pancreatic insulin content to humans (FIG. 2 A).

A correlation was found between the pancreatic insulin content and the dose of STZ (FIG. 2 A). By linear regression, a theoretical reduction of 30% of the insulin content would be obtained with a dose of 30 mg/kg as revealed by the histomorphometry analysis.

The pancreatic glucagon content was higher in humans than in native pigs and pigs treated with 30/50/75 mg/kg STZ (p<0.05) (FIG. 2 B) at 3 months post-STZ treatment.

Islet Structure after Isolation

Doses of 30/50 mg/kg were selected based on histomorphometry for comparison to native pig islets and humans.

a) Native Pig Islets vs. Humans

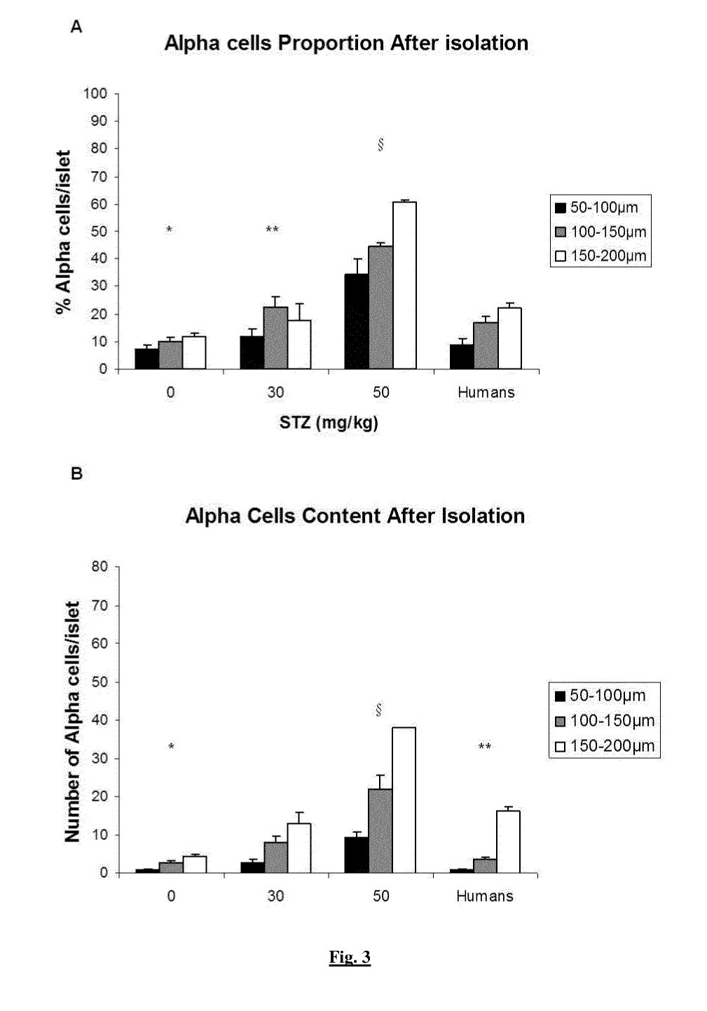

After isolation of pig pancreas, it appeared that native islets of 150-200 μm showed a lower proportion of alpha cells than human islets (p<0.001) (FIG. 3 A).

Large native islets (150-200 μm) also presented a lower alpha cell number than human islets (p<0.001) (FIG. 3 B).

Islets of 50-100 μm and 150-200 μm from native pigs demonstrated a higher beta cell number per islet than humans (p<0.01).

b) 30 mg/kg STZ vs. Human

Pigs treated with 30 mg/kg STZ presented a similar alpha cell proportion in comparison to humans in islets of 50-100 μm and 150-200 μm. They showed however a higher proportion of alpha cells per islets of 100-150 μm than humans (p<0.01) (FIG. 3 A). Pigs treated with 30 mg/kg STZ also showed a higher number of alpha cells per islet of 50-150 μm than humans (p<0.001) (FIG. 3 B).

These pigs demonstrated islets of 100-200 μm with a higher number of beta cells per islet than humans (p<0.01).

c) 50 mg/kg STZ vs. Human

Pigs treated with 50 mg/kg STZ presented a higher alpha cell proportion in comparison to humans in islets of each size (p<0.01) (FIG. 3 A).

Islets of each size from pigs treated with 50 mg/kg STZ also presented a higher number of alpha cells per islet in comparison to humans (p<0.001) (FIG. 3 B).

The number of beta cells was higher in islets of 50-150 μm from pigs treated with 50 mg/kg STZ (p<0.05) and similar in large islets (150-200 μm) in comparison to humans.

d) Native vs. 30/50 mg/kg STZ

Native islets of 50-150 μm showed a lower alpha-cell proportion than modified (30-50 mg/kg STZ) islets (p<0.05).

Large native islets (150-200 μm) showed also a lower alpha-cell proportion than islets from pigs treated with 50 mg/kg STZ (p<0.001) (FIG. 3 A).

A lower alpha-cell number was found in native islets of each size in comparison to islets isolated from pigs treated with 30-50 mg/kg STZ (p<0.001) (FIG. 3 B).

Treatment with 30 and 50 mg/kg STZ significantly reduced the proportion of beta-cells in islets of 50-150 μm (60.46±3.70 and 60.46±4.55 vs. 91.47±1.19, respectively) (p<0.05).

Native islets of 100-150 μm showed a lower proportion of peripheral alpha-cells than islets from pigs treated with 30 mg/kg STZ (p<0.05).

### In Vitro Islet Functionality

Pig islets treated with 30 and 50 mg/kg STZ were isolated and their functions were tested in vitro by incubation with different glucose concentrations and compared to native islets (0 mg/kg).

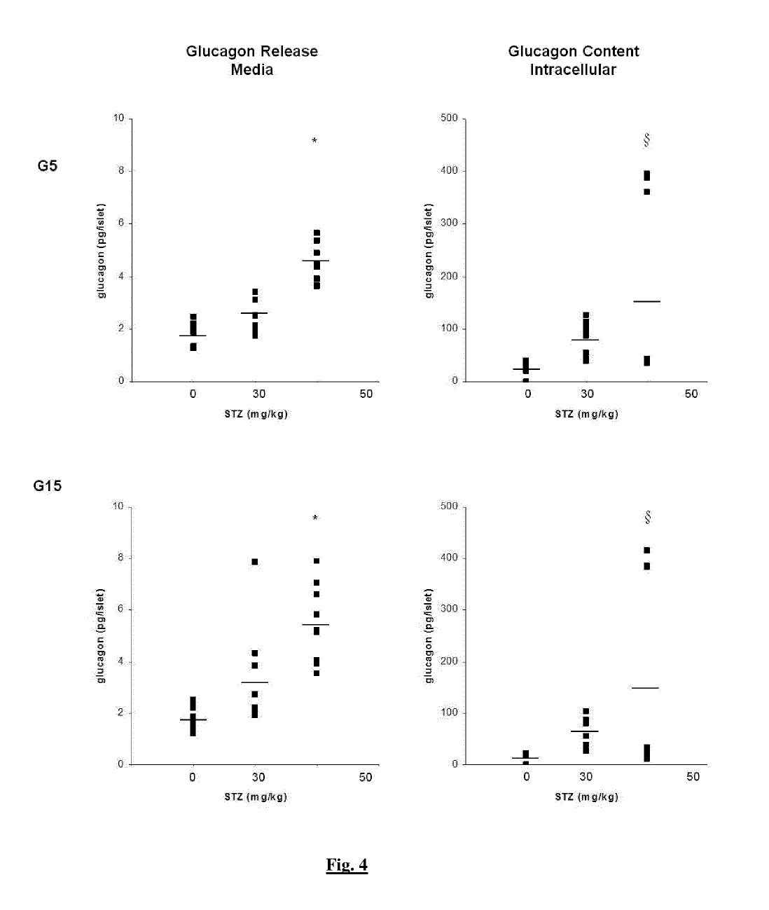

A significant higher glucagon secretion was observed after G5 and G15 stimulations of islets from pigs treated with 50 mg/kg STZ in comparison to 0 mg/kg (2.5× and 3.0×, respectively) and 30 mg/kg (2.0× and 1.6×, respectively) (p<0.01) (FIG. 4).

The glucagon content was 4.4 times higher in islets from 30 mg/kg treated pigs in comparison to native islets at G5-G15 stimulations. A significant higher glucagon content was extracted from 50 mg/kg treated islets than native islets at G5 (7.9×; p<0.05) and at G15 stimulation (10.4×; p<0.05) (FIG. 4).

No effect of Fsk on glucagon secretion or content was observed in both native and treated pig islets (data not shown).

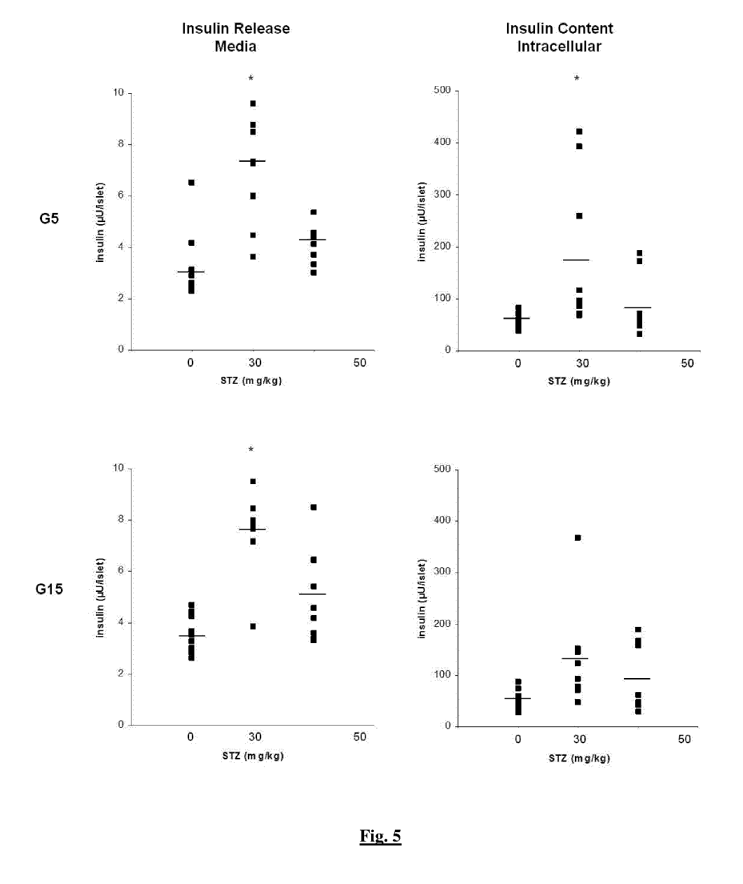

Islets from pigs treated with 30 mg/kg STZ secreted higher levels of insulin after G5 and G15 stimulations than native islets (2.1× and 2.1×, respectively) and 50 mg/kg treated islets (1.6× and 1.5×, respectively) (p<0.005) (FIG. 5).

Fifty mg/kg STZ increased the insulin secretion of 1.3 and 1.4 times in comparison with native pig islets at G5 and G15 stimulations, respectively (FIG. 5).

Islets from pigs treated with 30 mg/kg STZ contained 3.3 times more insulin than native islets at G5 stimulation (p<0.05). These islets presented also an increase of the insulin content by 2.5 times in comparison to native islets at G15 stimulation (FIG. 5).

Pigs treated with 50 mg/kg STZ demonstrated islets with insulin content 1.5 times higher than native islets at G5 stimulation and 1.4 times higher after G15 stimulation in comparison to native islets (FIG. 5).

No effect of Fsk on insulin content was observed in native and treated pig islets. However, a 2 to 4-fold increase in insulin secretion was found after G15+Fsk stimulation in comparison with G5 and G15 in native and treated islets (p<0.05) (data not shown).

### Discussion

Glucose homeostasis requires a complex control of insulin secretion by β-cells. A major problem related to the use of porcine islets in “pig-to-human xenotransplantation” remains their poor insulin release to correct diabetes. An increased proportion of α-cells within pig islets would (i) increase the glucagon secretion, (ii) leading to an improvement of cAMP concentration inside β-cells, (iii) inducing thereby insulin secretion in response to glucose stimulation.

To investigate this hypothesis, a pig model compatible with pre- and clinical application is needed.

In view to obtain an efficacious pancreas remodeling, the young pig model was chosen in reason of the endocrine pancreatic immaturity. Indeed, porcine islets are still maturating with a difference in the frequency and destruction of endocrine cells within islets between 5 to 24 weeks after birth and present therefore an α- and β-cell plasticity during this period (Jay et al., Xenotransplantation 6:131-140, 1999). Then, there is a possibility of endocrine cell mass growing and remodeling due to the high number of immature endocrine cells (both α and β). Moreover, islets from young pigs could have a better cell survival and a better cellular renewal in comparison to adult pigs. These latest pigs show also a low sensitivity to STZ and demonstrate no abnormal β-cell function after low doses STZ treatment. In this study, the reduction of the β-cell proportion by 30% after treatment with 30-50 mg/kg STZ did not affect glucose uptake as revealed by a normal IVGTT, in contrast with pigs treated with 150 mg/kg STZ or pigs that underwent pancreatectomy. It is correlated with no sign of diabetes after a destruction of 30% of β-cells mass in contrast to glucose intolerance and diabetes observed with a reduction of 50% and 90%, respectively. In comparison with rats and primates which become diabetic after injection of 50 mg/kg STZ, a dose of 150 mg/kg is needed to observe irreversible hyperglycemia in pigs. Pigs are also protected against STZ due to a low expression of Glut-2 on the β-cell membrane (Dufrane et al., Transplantation 81:36-45, 2006), explaining why low doses of STZ have no impact on glucose metabolism in pigs. Moreover, young pigs, possess also a lower sensitivity to STZ following their higher metabolism in comparison to adult pigs. The use of young pig offers also an advantage in term of sterile breeding and pancreas procurement (with controlled warm and short cold ischemia times) in contrast to adult pigs.

However, a selected dose of STZ must reprogram pig pancreatic tissue to obtain a human-like α-cell proportion.

It was previously demonstrated that, after STZ treatment, the glucagon content increased in hamsters (Dunbar et al., Horm. Metab Res 16:221-5, 1984) and the number of α-cells increased in monkeys (Dufrane et al., Xenotransplantation 16:152-63, 2009). In this latest study, an increase of glucagon mass by ˜89% after long-term diabetes (>53 days) was observed in treated primates. In our study, a linear relation was obtained between the dose and the proportion of pig pancreatic α/β cells. A significant increase of the number of α-cells per islet was found when the dose of STZ rose in correlation with an increase of the glucagon content in the pancreas. This latest increase of α-cell proportion and number per islet was confirmed after pancreas digestion and islet isolation. These results indicate that doses of STZ comprised between 30 and 50 mg/kg allow a reproducible remodeling of porcine islets with a decrease of ˜30% of β-cells and an increase of ˜300% of α-cells, leading to a “human-like structure” made of 60% of β-cells and 30% of α-cells in pig islet. In addition, no sign of β-cell regeneration (from duct or exocrine tissue) was found with no significant difference of β-cell clusters between native and treated pig pancreas. However, the origin of the growing number of α-cells needs still to be described. Alpha cells could be issue from (i) pancreatic stem or progenitor cells that reside within pancreatic ducts which can differentiate and migrate to develop new islets during both organogenesis and regeneration (Lu et al., Diabetes Res Clin. Pract. 78:1-7, 2007; Ramiya et al., Nat. Med. 6:278-82, 2000) or (ii) from progenitor cells located in islets (Petropavlovskaia et al., Cell Tissue Res 310:51-8, 2002). (iv) Following damages to β-cells (induced by STZ), these latest could be reprogrammed towards the α-cell lineage, on the contrary to a α-β-cell transdifferentiation described in the literature (Chung et al., Stem Cells 28:1630-38, 2010; Thorel et al., Nature 464:1149-54, 2010). (v) Finally, as the general shape of pig islets was similar prior and after STZ treatment, destruction of β-cells could allow α-cells to replicate and take the space initially occupied by β-cells. Then, the remodeling of pig islets structure into “human-like” islets must induce physiological consequences by improving glucagon and insulin release.

Although the pig islet remodeling by STZ is confirmed by histology and hormonal content, the improvement of pig β-cell function must be confirmed by a higher presence of glucagon concentration. After 30-50 mg/kg STZ, an increase of the secretion of glucagon (×1.3 to 3.0) was obtained by isolated pig islets. The effect of glucagon on the improvement of insulin release was previously studied in vitro by Bertuzzi et al. (J Endocrinol 147:87-93, 1995) and Davalli et al (Transplantation 56:148-54, 1993) on adult pig islets. An increased stimulation index by 30% was observed after acute exposure to 1 or 10 μM glucagon (Davalli et al., Transplantation 56:148-54, 1993). Moreover, islets cultured in the presence of 10 μM glucagon demonstrated a basal insulin release 2-fold higher in comparison with control situation (Davalli et al., Transplantation 56:148-54, 1993). In our study, islet remodeling with low doses of STZ induced an increased glucagon content and secretion which resulted in a glucagon concentration in isolated islets in culture of about 10⁻⁴-10⁻⁵ μM. This secretion acted as a cAMP trigger agent and induced an increase of insulin release (×1.29 to 2.13). The secretion of insulin increased also after a G15 stimulation in comparison with G5, with a G15/G5 ratio at 1.16 for native islets in comparison with 1.68 and 1.49 for 30 and 50 mg/kg STZ-treated islets, respectively. This represents a stimulation index increased by 45% and 28% for 30 and 50 mg/kg, respectively, in comparison to native islets.

In conclusion, a dose of 30-50 mg/kg STZ can modify in vivo the structure of pig islets, inducing an increase of the proportion and the number of α-cells per islet as well as a decrease of the proportion and the number of β-cells per islet to obtain an α/β cell ratio similar to that found in human islets. This remodeling induced an increase of hormonal content and release, improving by this way the function of porcine islets.

### Example 2

After isolation from wild-type pigs, islets were incubated overnight at 37° C., 5% CO₂/95% O₂ in RMPI medium containing 10% heat-inactivated FCS, 100 IU/ml penicillin, 100 μg/ml streptomycin and 5 mmol/l glucose. The function of islets in different culture conditions was assessed by 2 or 24 hr incubation of 200 islets in 1.5 ml buffer containing (i) 1 mmol/l glucose, (ii) 15 mmol/l glucose, (iii) 15 mmol/l glucose+0.1 mmol/l Forskolin (fsk) (Calbiochem-Behring, San Diego, Calif.) (added from a 1 mmol/l stock solution in DMSO), (iv) 15 mmol/l glucose+1 μmol/l fsk, (v) 15 mmol/l glucose+5 nM GLP-1 (Sigma Aldrich), (vi) 15 mmol/l glucose+50 nM GLP-1, (vii) 15 mmol/l glucose+500 nM GLP-1, (viii) 15 mmol/l glucose+1 μmol/l fsk+50 nM GLP-1. Three replicates per concentration were performed. Media were thereafter recovered for insulin quantification and islets were transferred in acid-ethanol for hormones extraction and quantification by radioimmuno-assay (Human insulin RIA kit, Millipore, Billerica, Mass., USA).

After the exogenous addition of GLP-1, a significant increase of stimulation index (ratio of insulin release at glucose 15 mM vs. 1 mM) was found for pig islets incubated with GLP-1 at 500 nM. Indeed, an increase of 10% of insulin release is observed for the incubation (during 2 hrs) of islets with glucose 15 mM supplemented with GLP-1 at 50-500 nM in comparison to glucose 15 mM alone. In addition, for a longer period up to 24 hrs of incubation with glucose 15 mM, a significant increase of insulin release by 12% was found for islets supplemented with 500 nM of GLP-1.

### Example 3

### Generation of Transgenic Pig for GLP-1 Expression

The development of pigs overexpressing cAMP in beta cells through the expression of porcine GLP-1 gene in beta cells by insulin promoter was developed.

The expression vector carrying the pig insulin promoter and the GLP1 (Glucagon-like peptide 1) was developed (see SEQ ID NO: 5). Primary Gal −/− and wild type fibroblasts are established from ear biopsy of the selected animals and cultured in vitro in DMEM/TCM199 with 10% FCS and 10 ng/ml of FGF in 5% CO2 and 5% O2. Growing cultures are transfected using Nucleofector (Amaxa) combining both smart electroporation and chemical transfection. Transfected colonies are then expanded and an aliquot frozen for nuclear transfer and the remaining expanded to perform PCR analysis to determine the integration of the transgene.

Oocytes are recovered from ovaries of slaughtered cycling female at the local slaughterhouse. Selected oocytes are matured in vitro in medium DMEM/F12 with 10% FCS in presence of gonadotropins for 42-44 h in 5% CO2 at 38.5° C. At the end of maturation cumulus cells are removed and oocytes with the first polar body are selected for further processing.

One method used for nuclear transfer is based on the zona-free system. Zona pellucida is removed by pronase digestion with a short incubation time till zona pellucida starts to dissolve. Zona free oocytes are stained with Hoechst and exposed to cytochalasine B before enucleation. Oocytes are layered in a row of microdrops individually and enucleated with a blunt micropipette. Oocytes are also prepared by conventional zona-enclosed method.

Cells to be used for nuclear transfer are grown to confluence and/or serum starved for 24-48 h to synchronise their cell cycle. Before manipulation they are trypsinised into single cell suspension and kept at room temperature until use. For nuclear transfer, cells are spread at high dilution on a culture dish (drop of medium) just before use, enucleated oocytes are washed first in medium containing phytohemagglutinin and then immediately dropped over a cell and rolled over till there is strong contact between the two units (Vajta et al., 2003). Subsequently the couplets (enucleated oocyte-somatic cell) are subjected to cell fusion. The couplets are transferred to an anionic media containing 0.3 M mannitol, 0.01 mM Mg, PVA and then to a fusion chamber. Fusion aree obtained by delivering a double DC pulse of 1.2 Kv/cm for 30 μsec. Couplets that do not fuse are re-subjected to a second round of fusion. Fused couplets are activated within 1-2 h after fusion by double DC pulse of 1.2 KV/cm for 30 μsec in the fusion medium containing 1 mM Ca and incubating them in 5 μM of cytochalasin B in mSOFaa medium for 3.5-4 h. After activation the reconstructed zona free embryos are cultured in the modified ‘well of the well’ system (Vajta et al., 2000) in microdrops under mineral oil to prevent adhesion between embryos. For in vitro culture 20 μl microdrops of mSOFaa (Galli et al 2003b) under oil are prepared and then 10 to 15 small depressions are made using a blunt small metal device. In each depression one embryo is accommodated for all the culture period. On day 3 of culture half of the medium is replaced with fresh media. On day 5 embryo development is evaluated. Compacted morula and early blastocysts are transferred to the uterus of synchronised recipients. Pregnancies are diagnosed by ultrasound on day 25 of gestation. Recovered fetuses or newborn animals are subjected to analysis to determine transgene expression in the islets. The pancreases of the foetuses are analysed.

1.-14. (canceled)

15. An isolated modified pig islet capable of producing glucagon like peptide 1 (GLP-1), or capable of producing higher levels of glucagon than a native pig islet.

16. The isolated modified pig islet according to claim 15, wherein said isolated modified pig islet is:
capable of producing GLP-1; and
a transgenic pig islet expressing the GLP-1 gene.

17. The isolated modified pig islet according to claim 15, wherein said isolated modified pig islet is:
capable of producing higher levels of glucagon than a native pig islet; and
a transgenic pig islet overexpressing the glucagon gene.

18. The isolated modified pig islet according to claim 15, wherein the structure of said pig islet is modified to increase the proportion of glucagon producing cells.

19. The isolated modified pig islet according to claim 15, wherein the proportion of beta cells compared to the alpha cells ranges from about 2.5/1 to 5/1.

20. The isolated modified pig islet according to claim 15, wherein the proportion of beta cells compared to the alpha cells ranges from about 2.5/1 to 3.5/1.

21. A method for transgenic modification of a pig islet, said method comprising the use of a vector comprising the nucleic acid sequence of GLP-1 or of glucagon.

22. The method according to claim 21, wherein said method is carried out in vitro.

23. The method according to claim 21, wherein said method is carried out in vivo.

24. A transgenic pig comprising a modified pig islet according to claim 15, wherein said isolated modified pig islet is:
capable of producing GLP-1; and
a transgenic pig islet expressing the GLP-1 gene.

25. A transgenic pig comprising a modified pig islet according to claim 15, wherein said isolated modified pig islet is:
capable of producing higher levels of glucagon than a native pig islet; and
a transgenic pig islet overexpressing the glucagon gene.

26. A method for obtaining an isolated modified pig islet having a structure modified to increase the proportion of glucagon producing cells, wherein pigs are injected once with 30 to 50 mg/kg of the pig body of Streptozotocin and wherein modified pig islets are isolated 2 to 6 months after the administration of Streptozotocin.

27. A device comprising isolated modified pig islets according to claim 15.

28. A device comprising isolated modified pig islets according to claim 18.

29. A method for treating Type I Diabetes Mellitus or Type II Diabetes Mellitus in a subject in need thereof, comprising administering to the subject a therapeutically effective amount of modified pig islets capable of producing glucagon like peptide 1 (GLP-1), or capable of producing higher levels of glucagon than a native pig islet.

30. The method according to claim 29, wherein said isolated modified pig islets are:
capable of producing GLP-1 and
transgenic pig islets expressing the GLP-1 gene.

31. The method according to claim 29, wherein said isolated modified pig islets are:
capable of producing higher levels of glucagon than a native pig islet and
transgenic pig islets overexpressing the glucagon gene.

32. The method according to claim 29, wherein said isolated modified pig islets have a structure modified to increase the proportion of glucagon producing cells.

33. The method according to claim 29, wherein said isolated modified pig islets have a modified structure wherein the proportion of beta cells compared to the alpha cells ranges from about 2.5/1 to 5/1.

34. A method for regulating blood glucose levels in a subject in need thereof, comprising administering to the subject a therapeutically effective amount of modified pig islets capable of producing glucagon like peptide 1 (GLP-1), or capable of producing higher levels of glucagon than a native pig islet.

35. The method according to claim 34, wherein said isolated modified pig islets are:
capable of producing GLP-1; and
transgenic pig islets expressing the GLP-1 gene.

36. The method according to claim 34, wherein said isolated modified pig islets are:
capable of producing higher levels of glucagon than a native pig islet; and
transgenic pig islets overexpressing the glucagon gene.

37. The method according to claim 34, wherein said isolated modified pig islets have a structure modified to increase the proportion of glucagon producing cells.

38. The method according to claim 34, wherein said isolated modified pig islets have a modified structure wherein the proportion of beta cells compared to the alpha cells ranges from about 2.5/1 to 5/1.


SEQUENCE LISTING (9 sequences)

SEQ ID NO: 1 - DNA, 6202 bp, Sus scrofa
atgaaaaccatttactttgtggctggattgtttgtaatgctggtacaaggcagctggcaacgatcccttctgaatacagaggagaaatccaggtattaaatcttcagatttgaactaatatagcactatagttagaatacagcccaggataaccgtaactcatgattaggtcttggcaatttagagtaaattctccgcacaattttcaaatgtttaaagaatcattgtgataaaggctttactggagtatagatcagagtatgttgtctcataatcaaaagggtgggaaatggtgaaaaatgtgctacatgggaaattatagctctttcaaatcttcagctgatcctacctgtccattcattattcacactgcatagtagatcattttaattattcacacaccaacttttccttttttattccttttaaaaagtaacattgttgctttctaactgaaaaagtcatacagtcataatagaaaatatgcatgtactgaatacttatatatatttataaatgtatctcagatcatgtgtaactttgtattatgctatttctcttagcattatattgttagtttattcttatatcattataaatatctattaagtgtatgatgtttaatgcctacatactatctcatctcataaatgtccaatatttatttagctactcctctattgagtacttgagttcttcacattttcccacgaaaaatgtgaaggaaaatagcctttttccctacttttcttactattttctgaagattgatttctagtagcaagattatcgtgtccaagggattgaatgagtttaaattttttatggtctcttgccaacttgctttccagaaaggtcatacgcatgtacactccatcagcattatagaaatactcctctcacaatatcctccccaatatttaatccttgctaatttgacaggtaaaaatagtatctcatttctatttttgagtcaatcatgttgaatatttccccacatagctgtgccaaccaactgttgaagactggcatacggattatcttctacacagtttcaaattagcaagtgggcttttttctgtctttctttgaacttttcgttgactttccttcaccattagctaccccataagcaggtaatgaagctatttttttatactaatatgtgtgaaacagaatttggaacataaacctgccgccccaaaaagaatgatttggaattcttcacttttttttttaaatattcaaggcgttgctctcttctggaattattagcataattatgagtagtggaatttgttgaaaaaaatctatttaatccttttttcaggaaaaaaaaaggaagacagtttgaaaatcagctgctagaattccaaatcagagtgtattaaattctagtaaaaccatgggattcccatgacaaataaaggtctttctgaattgccgcctgaaatactcagtccacttgccttctggctttacgtgggtctgtattttgtggagatgtttctcaagtaaatctagtcttgaggagagatttagttgccccattactgttcctcatagatcattcccagctccccagacggaccctctcgatgatccagatcagatgactgaagacaagcgccactcacagggcacgtttaccagtgactacagcaagtatctggactccaggcgtgcccaggattttgtgcagtggctgatgaacaccaagaggaacaagtaagtgtctgaccctcatcagaaatttgcacacaaatgcagtacttaaatatgtctaaaattcctcctgactttcttaagttattgtgcatatcaaatagcacccataagatatttcagttttgatgattttcaagggcagtgatgctctcctttgtcttatgcctctaaatgtcccaccccttgcccctccaccctccaatgggacataaattttaaattgttttcccagctgcggtcttagcaatattcactctcttgcccatttttctttaaattcgaaagctgacttttttggacttgtaaggttaggaatttaattcctgatccaaagttaagagtaaattcaacattccctgtagggttcaaactcatgactgtgatcacattcaaagtatcctctagccaagttaatcatggtaaggaatggaaacagatgcagtctttttcttgtagaatacaatccaagatacacatttaaaattatagcctagctgcacagcatagagaagaaagcaaataagaacacaagcctatcgtaaggcatacattttaatgttggatcttctttgacagtgttgggggtttttgctgcattgaggctactggaggaaatcagccgcaagggtttcacggggatggtggaatttcacaatttctttggagggcagcccacaaggggaagggagttatgcatgcaaagtagttgaaggaaatctgaaaaacaagaaaagggaatagatttgtctggatagaatggagaagggaaaaggatggcggtggggagggaggatgggagaggagctatttccttccaggaagaataaaagagggtgattgaccaaaaattctcaaactggaaggaacaggaaagatggcgggtatggtcagaagacagtggggaaagtccctgtgaaggattacggatggagccattggtgaaaggtgagaaatcacaaatttgaaaaaatgcaaacaactgtcttcatttctggaaagagaatgccacaacctggtaaactaatgtaaaacaatagtagctccctggcagtttatgttgtgtttaagataaaatgcttcaggctaacacatgctggagtgactgaccatctatctggtcgaccaactccaccgagcactctgtacacacctgagggtgatctggggtcatcttgttagcattcctcattgctaacccttcacacatttcctttaatccgttatcttgtggtctttttgatgacattttgagatgctcctttgctttcccttgattcaattaactaactctgaccccataagtactaaaggcaagcagctattttgttcgcaagtaccctaacacagtggatagctcagacacattgagactgaagtaaaggcagctttaataattaaagacacagataagcaggggaggtgaaaatgggagtgagggagcttaaccctccttaattgttattggcacccctcccatcaaccttcactccagatacttaatgtagcttcaggatgagtcccctacccaccctcctctcccaaatcttcctaagccccttctacacagaatcctgaacccaaagctgccactgcttataggtgagaaccatattgctaaagagacagatcttcaatttaactttcacatttctttcaggaataacattgccaaacgtcacgatgaatttgagagacatgctgaagggacctttaccagtgatgtaagttcttatttggaaggccaagctgccaaggaattcattgcttggctggtgaaaggccgaggaaggcgagagtaagtctgtacattcttatttaactttttttttttccttgatgctgaaaacttagactacagttatctatatatagatctgcattacagaagcaattaatatagtcttacgaagtaaggaaataactccagttggtgggaaacagtttacaaaagtctcctctgttttatctttttttttttgtaagggccacacccatggtatacagaagttcccaagctaggggtcaaatgagagctacagctgccagtctacaccacagccacagcagcaccagatccgaacctcagacctacaccgcagctcatggcaacacgggattcttaaccctctgagcagggaccagggatcaaaccggtaacctcatggattctagttgagttcgttacctatgagccacaacaggatctccctctctgttttacctttgatagcaaatattaataatagagtcaagtaggttacttttcatcccccattatagttgtattataattagggtatttgtcagtccaggacaattttcagaataaatagagtgatttggtaactaaccaccccaggaaaataaactaggactgtcccagggaaaccagtgctaaaggaaaagaagaaacaatgctaaagacttagtttgacttcaaatatttacatataggctcagcatatatttatatgttagtaagtactcaggtaaagtgtgatgaagactgaaagaaactaaagatgtttaatgaaatgacattcaaaatacaaatatatggagacatccatatatttaacagtgtactcattttttatgggattgtcatatcatcacaaaccatcaaattatcgcagttgaaaatgaggctaagacctttaggtactttcttagaccttagcacaaccaaacagacccctgaactgtctcattttcttgtgaccattttgattgacatttgaattatgagaagcatgaaatttatgttcaactcttttttttaaataagatttaaaatgagcttattgagactttatagagctctcaagacaaaagggaactagtattgtcactgatttttaaaacattaacaatcaaatttacaaaatcagttgggaaaagtgtcacttatacttaagatgattttacagtgttttaaatggatacccgaaattctgctaaataaaccaatcaaagagaaaatctaagcgagactaaactttaatcccttaaaactctacttggcaagggttctgataaacactagttttacaggatataatacattgttctgaattataaacaaattaatcgtctcaacaataacttctttcttggcaatatattaaaatctttgggagttcccttcgtagtgcagcggaaacgaatccgactaggaaacatgaagctaccggttcgatccctggccttgttcagtgggttaaggatccggcgttgccatgatttgtagcgtaggtcgcagacacggctcagatctggtgttgctgtggctgtggtgtaggccggcggctacagctccgattagacccctagcctggaggcaatctccatatgcctctggggtggccctagaaaaaggacaaaagccccaaaaaaaaaaaaaaaaaaaaaaaaaaaaaaaaaaaaattctttgaagcagctcgcatctggaagagggcttggtttcaggtcctggctctgcacttttctacctactcaattccctttccaacagacaatttctgtgaggattttcatgatagaaacatatcattttctcaatcccccttacacagtacccatcacagccaattgataagcatttatttgaccaaatactgacattacctctctttttctcagtttcccagaggaagttaccattgtcgaagaactccgccgcagacacgctgatggctccttctcagatgaaatgaacactgttctcgataatcttgccacccgagactttataaattggctccttcacaccaaaattactgacaggtgattgtattttagttcattccgaaaaccatcaaaattctcataaatacgatcacactggtataggcctttcctctgtggggaagagagagtcttaactctcttggtgtataatcaaaattatgactcgttcaagagtacactattatatttgtttttaatcattacaactttattcattcacctatttattccagacacattttctgagtgtcagtaagggacaaggcattaggatcacagatatgaattaaaagtggagatgataagttattcacatattaatgccacattgcatattgctatgtatgttgaaattttagcaggaagtaaaatgtcagtatatatggtattaaatacttaatgtaataagcataacagtaaaactttaaatgccaaaattaaccacttatcttgattagctgtagttgctttttataattattcatagactttctgttttttaataggggaaatttaggataagtcatctttgaatgtaaatatgtgaagaacccaagacaaaggatgtataagatgaaaattttttatcaaaacaaatcactacactaattcctggataagttaactgtatcatctaggtattatactatgttaaatcattctctctctttctctttttttttagtctctaggaagtatgtcactaatcaagatcgtgttcac

SEQ ID NO: 2 - DNA, 2477 bp, Sus scrofa
  misc_feature (1020)..(1119): n is an a, t, c, g or is absent
atgtgtctgaagttttggggatggtcttcgatgttccatactgtagggattatgcaactcgagggaaggtaggaactgttcgagaagtgaagctaatacgcatcacgagataagaagtagaataaacgtacagttacttggcaggtgaaattagactcttgcagaccctgtaagcactgcctcttaagagagcagatctgatggtgttctttctcagtaggcaccaaggctctgattcccttcctaatgcaatgcaaaaccactttccaggcaaagaggatgacccctaaagccaggagaaatcttctgtggagtgagaaaagcatctaggctgaaaataggaaggacactaactggtagggatagaagtaagaaccaatttatatttatatttataagtaagaactaatatatttatcaactaattgttataatagcatttttccagcagagggcatactttgcattgaaatagatctaaaaaagcctccgaggacgagattttttttccatttgaaatttttaacgttaattttttgggcttctaagtggtttaaaaatattttaaagtatttaaaattccagaggatgttaaatatgaaatttatcagattcaagttaatatttttgcctttttagtaaactattcaatatgtgactgtaaaatgaggcagtttttccccccatggcttatgaactgatcttagaaactattattggaagcaaagatgttttcgttgtgttgtttacaaaattgttataggaaatcctgtcttttaattggacgaatattgttttccttcatttcaatagagtttgccaggaatgacctcttttcttcaactgatagaaattaccagcattttctaagccatttcatagctagctctgatattttccctagaggaaatgacacctatctaaaagttaactcaatgtattatataatctaactctaatattcaaagctcattcaagcagcaaattcctgagaatttaagatttctttccttgatctannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatccnccttacacagtacccatcacagccaattgataagcatttatttgaccaaatactgacattacctctctttttctcagtttcccagaggaagttaccattgtcgaagaactccgccgcagacacgctgatggctccttctcagatgaaatgaacactgttctcgataatcttgccacccgagactttataaattggctccttcacaccaaaattactgacaggtgattgtattttagttcattccgaaaaccatcaaaattctcataaatacgatcacactggtataggcctttcctctgtggggaagagagagtcttaactctcttggtgtataatcaaaattatgactcgttcaagagtacactattatatttgtttttaatcattacaactttattcattcacctatttattccagacacattttctgagtgtcagtaagggacaaggcattaggatcacagatatgaattaaaagtggagatgataagttattcacatattaatgccacattgcatattgctatgtatgttgaaattttagcaggaagtaaaatgtcagtatatatggtattaaatacttaatgtaataagcataacagtaaaactttaaatgccaaaattaaccacttatcttgattagctgtagttgctttttataattattcatagactttctgttttttaataggggaaatttaggataagtcatctttgaatgtaaatatgtgaagaacccaagacaaaggatgtataagatgaaaattttttatcaaaacaaatcactacactaattcctggataagttaactgtatcatctaggtattatactatgttaaatcattctctctctttctctttttttttagtctctaggaagtatgtcactaatcaagatcgtgttcacaacatcacctgccagccatgtgggatgtttgaaattttaagttctgtaaatttaagaggtgtattctaaagccatattgctttgcatgccaataagtaaattttcttttattattgtataaccaaaagattataaatggaataaaccattctcaaaatattactaaaatatcagctttaaaatatcaaagtgcagaattctatttccttcttattttggatgaagtatcctaacctgcttacatgtggccttgaaattgttttctattatataatttgtatatataaattgcaccaattgcaacatatctgcattacaataataggataaggggaggactggtagccacagttgtgaaactgcaaagagaactttcttcttgaaacgattatcatagaactggtcagctttcaaagataggcaaataaaactttctagctcttaa

SEQ ID NO: 3 - DNA, 22532 bp, Sus scrofa
  misc_feature (11527)..(11527): n is an a, t, g, c or is absent
gcccggggtcggaaggggcaattctttctggcttttctaccttgcttcttgtcccccctctcctttccaattgtatgcgggcgagtgtgagagccatggagcgaagagcctggactctgcagtgtactgctttcactctcttttgcgcttggtgtgcattaaacagtgtaaaagcgaagaggcagtttgtgaatgaatgggcagcggagatccccgggggaccagaggcagcctcggccatagcggaggagctaggctatgaccttttgggtcaggtaagagttcgcacttttaagaaactttcaggggcccaaggggactggcgggactggaatgttatctcccaagtgggagtcccaggttgcactctcttgagcccagtggcaaagagcaggagcttctccagctctcgcccctggcgcgagcttggagagagaaggcttgcattatttattgtttgggctgggcgcagccaggcagaaccaagctgctgcagctgggtagagcttattcttgctgtttgttgtccactctggaaatgaaatccgttcccgcgcagtttggaaaaaatccctccacgcttgttcacagaacttgagatgcacgcaagctcttaccaaccaagtattcctcgggagatgcagtgcgctctgtcccttgggactcagtggggctgcgtcaggagccgactgtttccttcttttcaatatggcagcatcagatccaagtcccgattctagggggctaagcaagcttggagttattcacagaagcccttgaggatatgcccagagcgttgggaatgtgccagaggccaggactcattacccacgggctgggtggaccgcggagtggactgggaagccagtgggagaagaggagggtgaaatacagggacccaactctctgtgcctctaccaggagctccggaatctcactgggaacttgcaaagtccgagcttaacaactttgattggtcagttcccctgaaggttcaagatgccgaagtgatgggaaaggatttctttctttctggttcctcagccaaatgcgtaattgctttctgggggtttcagcttatgaaggaaactgttgcccatccaagtgaggactgggttaacagatagctgtggggcaagagagggggaggggcattgaggctttaggaaggggtctgctgagctttgtgatttcacatgaacccgagggcatgtgagttttaagctttccttttccttggcttagaatctagcccgtcgttctttgcctagtaaattgttgtgcactgaaacggggggtggggggttagttgtaaaacattttggcgcgttttttctagaggtggtggtagagatgcagctacagtcacggatgagtaatcggcgtgggcgttggcgattccatagatcaatccgctggggaatgcgcctttccttgccagaggcttcattctccaaatttatgagccaaactgtctgaaatgcccagcaggaaagtcaatacacgaattaacttctcttctctgtgtcccaagaggagaccaagacttttgcttttacccagtccttgttctatgccagattcggcattaggagctgaagaataatagggtaatagtcaaaaaaaagaaacttgcaaggaatcaagacccaagagattggtttagttacaaatgactgcaattgaacataattggtgtagtaacagaagctgtttctggagtctccatacttgctgattgaagaattgaaaatggaagggattggggaggggtgaaagaacacagcccagggaggaaacgtttgcacagagtttaggaagaagagtcaaaggttgccatgggagcatgatgagattgaggaagccactctaggcacagaaaacagcttgtgggaaaccagggagatacaaagaaggcagcttattggtaagtccagaaacttcaaggaattgttgggatctaaagagtgagtagggggtgacattgcccttgtgggtaccctgaacatcatggaaatctcacctggctgtgagggcaaaaacaaaaaaaaaaaaaaacaacaaaaaaacaaaaaaaccaaacaaacaaaaaaacctggtgacttaatctttggggcagctaaatagcattgttaactgtgtggaaggagattggaggaaagagcaaaaaataaatccctctttcttttctcacatattagcccccagacatatccatacatggaccattcattatggtaattgtcttttaaaaatctcaagtcccaataaataaattataaataaataacaataaatcagtattcttggtattatcttttaggaagggatggcatagtcacaagaggctttagaatagagtgagaaaattcctttttatatttacatatgatttacatatgcacatttccagtttaaatggcataaataacaaatttttaaaaataattatttgtaaaagactgaaggtacttttatggttctgcttagtcctccctttgtttacacttaacttagcatcacaaacctgatgcttaacatcactatccttaaggcttcaagataaagctaggatggtgcataggggttggggtggaggggtccatcagaagcaaaaccattgctgatcttttcatgtggctactgttctctctccccagctccacctatagctcatagcagattcattccccgaggttcagtcttgaagcagggcctaagcatctccattaaaaagtctctggatgatgagaaccaccgatttaaagaatataattcttctgaaataacccattgtcaattgtctcaattaaaaaaaaaatacagggcagctagagtactttgtagattcaaattaaatattgttttgaataaagggaaactagcttaaaagaataacaatgttcattatccttaatgatgtaaagagttctgaatggaccaaatgtactgatagtcacccagagaagcttacacactctaaggtggaacacttgtctaaatgcggcatgctggatttttcctcaaagctctggcctatgggaagtcaaatggtcactaatagcagcttataacaattatcactatagattcaatggctgtattttataacaacagcaaattgtcctagccgaatgggacattgcctttatagtgccagatttcctttccacaaattaatgttctctgatggaacaaatttgacaaaatgagtaatttttttagaaatgtctacacaataatatcacccagaaaaacaaatgcagttcctctgaaatgtgtgaaacagaagtatcaccagctttcaagtatgctgtgagctcaagtaataatattgtgaggttcatctcactcaacacactgagaagagaggaaagccttctatcagtgccttcctagagaagaaaaaagttaacttcaaatatggggaatgggtttaacaaaagttgcctgcctctgttagatggatttcaatctaactcacatatacttattctagaaagaggatttttattttaattttttctcttatagataggatcacttgaaaatcactacttattcaaacataaaagccatcctcgaagatctcgaaggagtgcccttcatatcactaagagattatctgatgatgaccgtgtaagtgttttgcatttgtcttcaaaccagtaacctgatttacctgctttctttatgattgccaacaagagcaaaagcacttcaaatttgattcttttttgttgtaatcacaataatttgtctgtagtgtttccagagggttatatttgcacataaaaataactggattggatggaattggttgactaaatttttatatataaacatataatgtagatgaaatatatattatatgcataaaatgttagatcacaaatccagtctgtgagcatgaatataagtactgagaatcaagtagcagcactaatgaaaatgtttgacaatacatttaatcgatagcattttattatatgcctgttatgtaccaagcactttgatgttcattgtggggatgccaaagaaatataatatataaaccctgcttttaatagcattcagggtaactaaaaaaaaaaaatctgccatttatatcttatttggttatatgggaaactaatattttggagggagctttaaatccatgtcttcatgaggtaatgtttccataagtattataatgtcttacccaaaacattccatttctttttgtcctgataggtataatattcaataatcaggcaataggaagaaatatctaaaataaacataggtaaatttcagggccaatttcatgcatatctaatatacctactgatctttgaaatctgttttcaaggtcagcaaacatatccatagaaatgttgatttgaaatagaaatgtgaagtattggattttgcatatacaatgctgcctttgctcaagaatgtgccatactggtggaaatttgtcctggctggcttgatatctgtatacatgatttctgttgactaggatactctggagaattatggaccctttgtgagagataaaatacatcctttcaactttcccaaaagaaaagtgtaagtgacagaaaggtgctagaactgttgaaactacatggaattcatgaaccctgtaaaaaatttgctattatatttgttgtgcaactgatagaattttaaaagaagaagaagaaaaagaagacaaattatgagggtggggaaggactcggcctgggttaccagtattcaaacatgaaagaagtaatgacacccacaccaggaatgtgaatcaggaccatggacagagaccagctgctcacataacaccaattgctggcttttactcaagttgaaggctagatgtgaagaccaaatcagaggccctgaacattactttatacatttcttgtccagtgaaatcttccttttgctgtcccaaggcagatggttaaatgtatgtgggctctgaaagtaggacaacctccagcattctggctaggcatagtccttcacaaaacaagaggtatttccttgtctgcctaaatgcatgcctgacattactgcacaaggaagtcaaactgacctcccttccaatcttcataggagaagactggatagaatggagcagacctaactggtattattactcagagtatctcatataatgaacagatggcccagttttattctttagacatttccttaatagttgatttgggggatgtgtgtacaactgaatcactttgttgtacagcagaaattattacaactgtaaataaactatacttcaataaaacttttaaaaaagttaaaaatgtaggaattcccattgtggctcagcagtaacaaacctgactagtatccatgaggatgcgggtttgatcctgagcctcactcagtggattaaggatctggctatgagctgtgatataggtcacagacacagctaggatcctttgctgctgtggctgtggtataggccagcggctgcagcttctatttaaaccctagcctgggaacttccatatgctgcaggtgtggccctaaaaaaaaagccaaaaaaattttttttaatttaaaaaatagttgattttggggaaaatctcaaaacataatgaagaactaagaatttactctccagcataatgtcattaaaaaaaagagtctatatgatcctataaaaagttatccccagaataattttatttcagggaaaatcctagaacctatcgaatcttgcagaaaaaagtgggactagataatttttaatgtccttttcaaatcaaaagaattatttacaattaagtacatgatacctattatatacataatggaaggacaccaccaatttgttctgttccatttggtctgctcttatagagattaatacgggatttggagttaatgacattaatcataacactatttccaacaactggatatttatctgatcagatggcagagtgcctcttcttatcttccatttttaaaaaatgcttttgattttcattttattgctcttttttataggataatattctttcaaattagccttctgaaatacagcaataagctatctttataagtctgttaatgaaatctccaactgtaagtgacttttgccctcaaagattagtactcgttccctagcagccagctcccaaagaccaactcagatggcaggcagtatattcccagcactgaggtattttgagccaaaacaattaagcttgaaaacacaggcactttaaaaataaattatagtgactaatttctagctctcaagtccctcccaatgtttctaatagatttgcaatatattttctaattgaaaattcaggacagtcttagtggatggttttatcctttagttgcaaattatttttcagttttatttttggttctgactcattagtattctcacattaacaatatcctatcacacactggtctatcatcatgtatacaaaagagaggacaagttttggataggaaagagttatgttgcccagcaaaataagagcatggtattatctcataataaatataaccacaaggcagccccttaccctcttagggcctcaagtataataggattatgccataaatgtggggaaggtaatttttgtattgtcagatctctttgcataatacatgaaactaaattaatggtaagtgtgcatggtcagtagtgctcattcatcaccctggatctgtttctcatgttaaatatgcttaagcaggaggctgaagtacaggggaaccagaatttcaaatctgcaaagacacagaaccttttctgtcttttgtcacattttcgatttctatttttgtgtataaagaaagagagattaagctgtaagagcaccataacagcagtctctaggatctcttagcttgttgactcatcccatgaaaatggacaaattttcagctacagaaccctggaacctcaatataaaaatgggtccatttgctgatgaaagcaggctgaacttttacttccactgtaatcacagtgtaaataatttgaggaatcatcacttcactgaccacttctcctccccctaaaggtacactttactataatagaggaaatgctcatctcctcagtgggcaatcccttcatctcactgagctgtcagtttgatctgcagtcaaacgacaacactgtggagcagcctgaactctgatggacagggtactggagctagttaaagggaagatgattacactagaggtggctatttgactgttgattttcttgctataaaactccttgtaggtgatatgggctgaacaacagtatgaaaaagagagaagtaaacgttcacttctaagagactcagcactaaatctctttaatgatccgatgtggaatcagcaatggtacttggtaagtacctacagaataactgtgcaggacctgggttatttattatgactgcacaattgattttttttaaatgatggataattcagcagagagatgtattacctggagattagggttgatgggccctcccatcatttgaatcttccattttactaggtgagaatgtctgaaatacagatctttagttactatgtgaggaagggttgttgctttcttatcttcttaaatttatctggaataaatacagaagaatcatgtcttgcaagtgagagtttccaaaacagtactcatggcaaaaatcacattttatcaaagttacttttgagaacactttaaattacccaaagactacagtgaacataattcagtatttatagccaagtggtaattcttatgtgcttccaggtaagaatttctgatctaggctttaggaagaacacaaagaaaggcaaaatgcagctagctgcaagtgtctagacttgcaaaccattgtacctttaagatagttaccaagtccttattggaatgcaggcatcaagtggagaaattctaagtgttcctgcttgccggtagagtgctctccttctgttttaatattaagcctctactgtagtaatacacgttaataggagtatttgttgctgagaataatgtaaaagataaactttcatgacaaccataacctaaggcttagacatccttgttgcacagtgaatgggaaaagggatcaattcctcttgaaatattaacctaagcagtacttgctcaatgtagcagaagagactcactagctaacactggtccattactatgtgacaaacactattctaaatgtttgacatgaattaacttagttctcatgcaacatttttgttgtagatatagtaagtatgcccacttaagagatgaagaaatagagaacttagtaaattgattcacttgccaaaatcacacggcttctagcagttgggatcataattcagggctccagagactgttaacaaccacactaggtaattgtttttgtgagtgatagagaggacttcttctctggctttggccaagactgtgtgtagttctagactcctgtgactcagcttcatgctggtctagtcgatttttgttttgctccacctctttctgaggggcatcagccagggggcaatgcattcatttatatttttgattgtttttatttataaatttctttttgactccgcttaaaacctgcactcttctaggacgtatctcaaatgcttggggatgttctcatatctgtgttccttttctgtccagctcttctttcctacagtccacatactggaactatcaggaaaggtaactcgaaaaaatcctaaatctcctatcacagaacttactccctaaaccattcatactgagaaaccggcagttaaattgcaagtcctagtgagaccacagcctcttgggtgttgcaggttctacttttttcttttagaaaagttataaaaacatccatgaataactgtgagccagaaaaaattaagtatgtttggtgtggccatggaggctagggatttgggggcagatggaaacggggcatcaaataaagctctggtaaatgaaaagctcgagcatgtaattaaaatgaaagcaacttagggataaacttcttcaaagccacatagctcaaagagaaggggtacagggtgattcatcctttcaagtgaatttccattggaacgattggaaaaagactagcactatgatgattcacttggtcctaccctcaactttttactttttttttctcacttaattgccttcttctaaagagtagagtgaccctaggaaaaagaaagatgggtttccatactggagcttcagttctcattctgcatcaagcaaatcctgagtttaccaatgttccttttatttctttactagcaagatactaggatgactgcagccctgcccaagctggacctccatgtgatacctgtttggcaaaaaggcatcacaggcaaaggagttgttattactgtactggatgatggcttggagtggaatcacacagacatctatgccaactatgtgagtctgggccctcccattgctgcaacttagggagggcctacacggttaggacattccttccctcaagcaggaggctgctctcatgtcatccctgggactgatggttcaaagttcttccactttcctcaccctcctgccagtgcacataaactttctttctttctttctttttttttttttttttttttttggtatcactgggcattgaaataattcagtggagcccaggattatacaaacacatgtgctttgggtgaaaggaggcacttaagccaatgaaattattaaaagccatttatctctatgccactaagtatttggggacatgaaaatgatcaaatcaagccaaatgcacagtcttcctcctcagtcccttatggctcaatatctctcacattgtaaagttatatgaatgtgattcctttcatgcacatcaaagtatataccacacaatgcaaaaattaacaagctgggaaaagattactaggtatgggaaatcaataaattaaagttatttacttaattgtttatgaagctctccataatgttttaggtctttagatcagtttttcctcttgaaaattgcctctcctctttttctcatcccttccactcaatcaacattccccagctaaagagcaaaagaaacaggaaggcaaagaaaagatgaaaatgattaaagaaagtaggacttattgaaagatttgaaaataatagagaaatagcaatagcgttaatacagttcctatgtggaagatgttaagtatagcaatctgattgaaaaagggggtgggacctggaagctagtctttaccctctttagttaaattacatgtttatttagtgtaactggggatgctgaaggagatcacttagaccagagccttaccccaggctccttgcacctatattcttaagaaatgagtgtcaaagtaatgctaactagttctatagctgagttaactcattccatttttgtgggcttcaatttttcatttggaaaatgagggggttgaactacataaactctgactccttctggttctaatatcttatgagcttctatgacctatggctttggtaacattcaccgagaattggacatttgcttgcttttctgtgtacatcatctctgcctggttgatgggtataataaatgatcagcaactgggccagaaactctgaaatgcaagctttccggttatcaggtagtatggtaatgggacagggctagaaggcttggaaagtactgctttttgaatgatgctatggtgttatttttgttttttttttttttttttttaggatccagaggctagctatgattttaacgataatgaccatgatccatttccccgatatgatcccacaaatgagaacaagtgagtaaatgtgggttgggggcgggggtaagtgcttcaacatgcttttatcttgtttgagaaaattacactataaatatgatccgcattttagctctcagaaatacaccacccaaaccacaacatttcgttgcttttaaaacagaaacaaaagtttctatttttatattccttttggcatatagaaccatgtttcaattaaactttcttcttatattatgaaatgcttccctcaaatgttattgaatagttatagctttagaaaaaatgtttgggacataaagaataaaaacctgggggtcatcctcccggcccacaattcctgtataaatcttttataatttgtaacatgcttgatgttagtcatgaagagaaatacagataactacgataacacatccccatgacttcacagagaagccacaaccatcctcactagtttccccagaaaagcatggccagatatcagaaaagagaaatgtatactgaagcctggccttcgaatcccacacaggctccagggcattgggccagatagtagcctattccggaattcctgctggatattcgaaaatttaagagaagagaaaaagtatattttctgactcctaaattcactctccatagcagccacaggatactgaaggagaccaagagccaaatgtgccanacaagaggcccaataaagacccagaagacagagatagtatcacctttctttcctagacccaaaagaaacttcacccctaggtactttcactcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatcacctcactctgcctatatttttttattttggttcctataatggtgactctgtccctactgtcctaaaagaacttcctaagaaaatagggaataagtgcttgaaagaaagggatgccattcttagaatgaagttacaacaactctcattctacctcccttggtcaagattaagagggtaatctgctctcagccatgttagtacagctgaggagactgttagctggcggtatcaacctcactcctaggcagtgtgccctgggtttggtccaggatccaatgtgggtgataacttgtgtggtagatccataataagtgaatacctggtgaggtgatattctctagagtctttgtactcatgagaaggggaattcagatgtttctcttctttccccttaaaaaagggattctcaactcatgatacttaaaataccacatttccccaaactcaaagagttctagagaccccaaggaagtagctcaaaattgtgtcattttatgcctgaaaactctgagcctccagagcatagcagaccacaggcctttcatatagcatagaagtggtatgccacagccccatacttaagagtacagactttgcagatacatttggatcaaatcttagatctacctcttaatagctgagtgaccttgagcaaattatataacttctttgtgcctcagttttatcatctactaaaaagaggtaataatagtatctatgtcattggattattacggggactaaatgagtttatatatgtaattagcttgcgtagtgcctggtacataggaaaagttaagtgtttgctatgatgtcgatgacagtgacaaagacactaatgatgatgatgatcaagtcctctcatttagtaactaaagtagacttcttttcagtgagaaacatgacaagaataatatctgataagaatagaaacagagacaagtcaattttctgagccaacttggaatatgctattgaaaattacacaaaaaatacctctcatggtcctcttgactatcaaccaaaaaggtagcatctctgtcatttatcaaaaggtatagtgaaaaatacttgcccttctagcatgttgtcagaaataaatcccttacatttagaaaatgaatgaccccattttgtcttgatcgttataagcaattagagacctattatctcccaattccaaaggtgttgtctctccaaactatctgtgttgctgctgttctttattactatgtaaataaggttaaaaaaagttgcatttgaattctcttctgcaaatccaaagagaaaacactaaataataaaaccttaacagtgcaactggcatgaatctctattcagtatttcaacaccaattacagattatttttctagttaaataacatgttgtgaggatatttttaattggtacaataatagtttttaaactatctgtggaataatttttttaaatcaatttgtttaaaagccagttatggctctcattctaagataatagaggttcttttatctcagagccagatttaaggaaagaaatctgcaggtctacaaagaatgacaaggaaccactttgataaagtctctttttctcaaggtgcggaggtgggaagtgaggataagaggaggaaaatatatcaacccctatttgtatcatgaatctgtaccttttaagaatagagtcttcagtatgtaaagagaaatcaataataggaggtgtcacattgcatcatttttaggaccagaggtacaatgctggggaagtatgccaacaggttgattctgtaagctctgtctggagactgtgctatcatggtacctctctccaatactgtataaatggaaatacaggtggtggtgacttccagaagcacatttcccagaggatggcaacaatttttttccttccaacaaaggcaaagaggactaatccatcaaagaagcataaagtgccatgctgctatgcgttcagtcattgcaagatcaaatcatatgccatctggacacaaattactccatctctatctcctcgttcttcttacttccacttagctttattctcaatagactctatggtagcaagagtcacatggcctacatggaaattaaaggcaaatattctggaaaaagagtctaattctcttccccagaatctgcatcaattctcaaaagttgtctggggtaaaagcctcatgaccgaaagagaatcataaggagtgagggccatttcccaaaagggggaaatgctagaagaccaaaaagaagcagatgtagagaagacctcaaattaaaaatactcattttgggagttcctgttgtggtgcagtggttaacgaatccgactaggaaccatgaggttacgggttcggtccctgcccttgctcagtgggttaacgatcctgcattgccgtgagctgtggtgtaggttgcagacgcggctcggatcccgcgttgctgtggctctggcgtaggccagtggctacagctccaattggacccctagcctgggaacctccatatgccgcaggagcggcccaagaaatagcaacaacaacaacaaagacaaaaaaaaaaaaaaaaatactcattttgttctgaattgactcctatccagtattaacaaaaaggaggaacatcatttggtgaaagaagaccaggaaattatctttggttttcagggaaaataagaaaagtaaaacaggcttattttaaatgacccattcaatttggggtttttgcatttcatgctgggctaaattaaaagatgtctagaacagtagtgtctaaaagtagctggctgacagagttgctcagagttaaccagcctatctggtcagtggctccactgactgctaccagatgcatgcaccatggaataaaagtcatgaaatatacaagttcacattaaagttctcttgacttattgaccctcctgaggtcagggagaagatagaaaacaatcactatcctcagaacgtcagaattgatgctttaatggtggactttggtctgttttagagatggatttttttccccaaaatgctctgcaataataattaccttctttttcttattgcaaaactagtcatgttcaggcttctgttcccaatcagagttgcaggagaaccatactttcctaatagcctgtagtatcttgttaaaatctcaaatccttggtaccagaaaatcttcagatgttttagcttggtgtatggttagaggaggagtggagggtttgagaggggactctcttctatatattctattaggactggtccaggacaacctaatccaaggaagtcactagtgagaaagaagtgaggagtcactttaacagcaacgaagtatggtgcagaagccatttctttgtgatggtttctgaaagtgtcagagctattcatagtaagtatcaatagctaacatatcctgggttcttactacataccagccactgttctaagtattctcatgtgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcagaagctagaatttgaactcatatacttgggcccttcatctgatacctacagttcatctaatactgatgagaggaaaaccagctacaaggctgtcaaaatgttaatatatccatgtgccatagattcatatgcaaaaccaatgtttttcctcatttatgttttataaactttattttttaatcttcagacatgggaccagatgtgcaggagaaattgccatgcaagcaaataatcacaagtgtggggtcggagttgcatacaattccaaagttggaggtaaagcagagggttgttcctgtagttttgcactttaatttgactcttgcattacctggtgtaaaggtgatggggtgaggtggggtgggggcacttctcaacaaccagaggaaaaaatgaactcctttcaaaatttgctgcatgacttgaatgtgttttgcaggctaatcaatgaatctggattcttagaaaatggtagcctgagtatagccaatacttctttgtaggggttaggtagaatagaaatgttgaatagcttttgaagcaaatttaattttctttgtgttctgatttagaagggtttgttattgagtgcttgctaagagaacagaaggaggaggtaaatttaggcaaagacttacattgcctctcaattctacagtcccttattttatagcaaagaaaagttttttaaaaataaaaagaatacctactcattatagtaagaagaagaacgatatctatgagagcaagaatttggaatctctggttgttttcatcagtgtctttatgggtaaacatatagtaagtttcacagggcaatttaataaaagaactattgacaagaattaaaggacaccaaagaggcactaattaacacaagcagatgttactacctataggcctgatggagcaaggggaggacgtggtcatggaaactagagagagaatctgtggccacaggagacagccactggacaggagctatggctttgagtacaacagtggagctgcttctaggcagggaagatccaggagtgagtacctcactcttgccccacccccaatctcctgccgctgtcttcagttggctgaaactaatgggatgacaagagaagccactgctgccatccaccttcctgtggagaagggtgcagagtgaatccccaggggctcacagagaataaaataattgtctctcctcttcacacgagccctaagccctacctcaggcttcttggttactctataaactttacatttagtttgttttctaaaacacaacaacattgtagaacacagcaaggacagtaaaactccattaagctctacaaaagtagaccttgaatttggcttcatatttcttttcagaaatcaaatatatatacatcctggtataaataatgcatctctcactaatacagcacactcctagattttaagtgtcatctctgattcttttgataccatcatcatcaaagtgccatgttaatttaaagaaaatgaaggcaggaccacaaagtacataatcatctaggaaaagaatgtactttttaagtcatgatgaactaagtagctatagtttgtgagagaatcatctcatcccttcattttatagaagaggaatcagaagcaagatttctcaaggaaactaaaacatacctactatatgtccatcatttatgttacaagaccttataggcagtgtaacctagtggttaacatcatggatttgaattctggatttctgagattcaaggctcatttctccagttttgagctgtgtggccttgggcaaatcactgtccctatctggtcttaacctctctccctttaaaatggggagtattacagcacccacctaagatggttgtcataaggatagactagttcatacttttaggcctgagaatggttgctacatatagtacatcctgaatgaacaggagctgttatcatccccattttacagttgaagaaactgaggcacatagaagttaagtggcttgcccaagatcgcagagctagtacatagcagagatgggattttttcccccaaatacattttgaacaattttaaaaaataagtatgacttcaactagtcatgtcaactacatgttcttttacctatgctccagagtaaataatagtgtgaaaatctaaggctgtgcactccattgcactattagcctgcagccaggaataaccaggtagatgcctatatctgtctaaattgcttttgccttcctggcccttgaatacatgagtctgatgatgggtgacagcctgttggaaagcacaaaaagctaccgcatctgccttcccctgatgaaaaatgattccccctgaggagtgaaatacatggcaatgatttcatccttgagattatttagaagaaaaggaagcatgttccttgaaaatgtttcttagcaaacacataaaagaatggcctctgggttatttttaatcagtgcttaggaacaagagttctttccctatttgttagtttcctattgtaagtaggtcttttcaagctgcagagcttcctgtgcagaattctctctaagacactgtctgggccacagccatttgtggtagagttatgagcagacatgttgaaagtcggctgttatgcctctttctgaaagagatcctgtcattttcaggttattagggcagcagagggaagggagacatttcttaaccaaccacaactatggccactgcccagcgcatagagttacctctaaagggcagataagaaggcttatggagacagtgactagtatcttcctgccatgctgttagcagctgggtcacaggcatgtgggacatacaatggttaccctgtttttgactctttggggggtccttttaaaacaattattaagagcattgtgccattctacatatacattgacactgatgtatgtatttgtctattttgctgttcaggcataagaatgctggatggcattgtgactgatgctatagaagccagctcaattggatttaatcctggacatgtggatatttacagtgcaagctggggccctaatgatgatgggaaaactgtggaagggcctggccgactagcccagaaggcttttgaatatggtgtcaaacaggtgagatgcttaaaataaacccacttgaagtgaaacttgactgtttaaatatggggggaagggaagaacacttagtttatatgcacagaaatggttatgaatttcttgttgtttatgatttgaactgcatttttttttgttttttgtttttttttgtctttttagggccacacccacgacatgtggaggttcccaggctaggtgtctaatcggagctgtagccaccggccacagccacagccacagcaacatgagatccaagccgtgtctgtgacctactccacagctcacggcaacaccagatcctaaccggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncacctcctgatctggcttttaaaaagcgaaatcattctggggagggatggaagttaagagttaagagaagtttttccagaatgattctacatttagactgatattcatgtcccctagaacagtttactttcaaaaagtactccagataaggcctttgtcattttcctgctgttcttggatttctacgctaaggaaactttcacccagacttctttcaaatactctgaaaatgcatccttaactatagcaatgtggcgcttccctctctgcacgtgcctgatgattacttgtgccttggctgtgttgctctgtcactgaggtttaatgcattctcgccaggattctaaggattctgtgcaggatgaaataaaagtagaaactttggagagcacaaattgaaaaatcatagaaagagtagccttgacgagttccattcaattgaagagtcccttttaatgcagctgcaaagcacatctgaataatgcctgattttgtcattctatcagtcagccagaagattcaagcagaaagaaagagaggaagttcaaagattcaagcatagtgacataagccgagggaagagtgaaaaaattataagcatatgctataagccattatgaatccacataaaaaagaaaagaaagagaaatactcactcagtgccattccagaaagggtggggatgaaggaggctctggcagctccagtgcccaggggcttagaacatgcagtctttcgagagtttcaacatagatgctggagtacctggagtggaaacagactccaagttaatgtctctgggaacttcctgcatctatttgttagagattttattggcgtgtgacacggaccccaatctatgataacagatgtgatataatctggcttttcctttaggggagacaaggaaagggctctatcttcgtctgggcttctggaaatgggggacgtcagggagataactgtgactgtgatgggtacacagacagcatctacaccatctccatcagcagtgcctcgcagcaaggcctatccccctggtatgctgagaagtgctcctccacactggccacctcgtacagcagtggggattacaccgaccagcgaatcgtacgttgcacttttgtctaattctctctgcttatgtaggagagcatggcttgtcttcaaaaggatcctttggttaaattggtttgcttaagcttgatgtctcagttggggagactcacattttactcacttgaaagaggagaatatattgggaagttatctctctcagggataatgacgctactgccaagaaaatgctgcctctctgtcatttctaaactcagccctgagaagccaccaacctgtcattatattcagtcgagactccaaatgtctgttcagcttgtgtgatgcctccctgaaaaccgccttaaaggagatttcagagttgtaggggctttatcacccctgtacttgttgatcctttaatagcagggaaaatgcctgacttgccctttaacagacgataaaaatacagcgcatacaccaactatgtctcctaagaacggcgacctgcagaaaaataacagtctaaatttcccccgagatgaacttctcagcatccacataagcagctctggaaggttatatgctcagagagcatctccagcccacgcttggctgtgccttcacttccatgagtcagactgccaagggcaattattggatttgccttcaagggcatggtgatgaagggctattcgggggctttgaaaacaaaggcagttttaatttaaaaatgtaaagtttcaaatttaatgctgacacagaaagaaccaactagctaaaggatagagatgctaatctcaataagataacagatcatgatccagttagattattagaccccctaataatcagcccttggaatgtgtgtaaaattcatattaatggcatctcaacaaaactgtcagaactatgatctatatactggatcactattctagaactctccctgtttctacaacccactctagtttagacagtaaaattgtggtctcaacttagaagccctgatgccctctcatgcccacatcgccatttactcaaatagacaatgacattctaccaggggttctttaaggcaggcatatcttgtagcctttgaaaggcgcctctgggccatgtgccccacttgcatgcacagcccctgactcttgatgtccctgtggatgtccagagtggcttttcagaacatggcctgttgtgcatttcagagtcaccgtttcatagactatctctcatctgagccactctactctgcagtaggaggaagattttccatctttgtagagatcatccctccttaggtaaatcagccacagaagagacagctggtggattctatttgagacagttgcaataacatagcctttgtcttacgcaaacctcttcctcagcagagtcatctctgggggcttacttgcgtcaacaggtaaaaagggcaagcttgtttccgcagccctcggagaaacagagccaccataagttgagatattcagcaactccaagtgggaagccaaaggaggtgataatagaacaaattatttttttacacctccctcccacacattgaagttgctagctaaaaaccatagtgtgagtgcgtgggtgggtatcttgttagtaatcctttggcctaacttacttgttcaaccagcaacacagtggtccttatggcagactaatgggaagtctgttcggtttcttccctttgtctcagcagcctcttcattaagctaaaattagcctgggtaaatccttatgtgcagtaacattgcgtgaaaatgctttcacagacaggatctcacttcagcttcagaatatgagtcaagaaatatcaacctcatttgacagatggggaaaccaagtgacttgcccatcggacacatctactgagtggcaggtttagaatgtaagcctggatctaagttcagcacactttcacttgaaaaattcaccattttcatccaagttcaaatatgacactaactcaagaccatcagattacatcaacagtgagtgcaaactacacagtagacattgcaagtaagtagttcaagtggccagaatcaggtcagctttacatccttgtgctgaagaggcagtcaaatgtcaatctaaaggcaaaagggaacggtgcgtgaggcagaatttctaaatatgccaggccaaacacaatggacctgatgattaatgggccagcatgattcacagtgatttttgaaacagtcccttttttacagacagcatttcgtttgggaatctcagaagagaaaatattcacagtgggagtacttcatccccagaaaaggcatcctcatgccagaattatagacaagttgcatacagaggagagtgagctaaaatatcagcagatggcaaacatccgcctgtggtttggaggaggaagtagggaatcgaatcagctgggcttcctgatcacagcagatgcggccctcggtagggttaacccccggggacaggatcatactcctcacatgttctcccacgtttctttccttcctggatgcttcccagacgagtgctgacctgcacaatgactgcacagagacccacacaggcacctcggcctctgcacccctggctgctggcatcttcgctctggccctggaagcaaa

SEQ ID NO: 4 - DNA, 699 bp, Sus scrofa
atggcgcacgctgggagaacagggtatgataaccgggaaatagtgatgaagtacatccactataagctgtcgcagaggggctacgagtgggatgccggagacgcgggcgccgcgtccccgggggccgctcccgcaccgggcatcttctcctcccagcccgggcgaacccccgctcccgccaggacctcgccgccgccgaccccgaccgcccccgccgccaccgccgccgccgccgccgccgcggggcctgtactcagcccggtgccacctgtggtccacctgaccctgcgccaggccggcgatgacttctctcgtcgctaccgccgcgactttgccgagatgtccagccagctgcacctgactcccttcaccgcgaggggacgctttgccacggtggtggaggagctcttcagggatggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcatgtgtggttgaatgtgagtctgggctgcgcgttaagggtctgagatgcagtggttggagtgtgggtgggctgctcgaccaagggcgggcggatgagccagtgctataa

SEQ ID NO: 5 - DNA, 12612 bp, Artificial Sequence
  Synthetic Primer
gtcgacgagttcagctgagctggctcccaggtcacctctctgggtcttggtgcccccagcatctcccaggctggccctgccctgggggtgcccttcccaccctgccctgggccttgtggagggcaccctgggctcactgggaggcggtcggccctttccttcccgcaggatgtaagcaccagcctatcttccaggccctgcgccctccctgggtgcccccctcctacctccagagccctgactctaggctcttaggatgtcggtcttggaaaactcctactcatccgtcaagaccctcctgggaaaacccttccttcccagccccccaccctggatctgtgccctttcagcctttgaggccacaaatgaggctgtttccaaaggttggaggcccctgggaagggctgacggccggcctcctcccctccaacccctgggccctgggctctgccctcatccagtctcctgccttgcacaccctctcatagaggcccccagatcttccctggctgcagacgggcctcaggaccccctgctgtcctgggaagccagggcccagctccttcctccgcgtggggtggggcctccccacaggggcctgtcccgggggggtaccagagggtcacccccgcacatgggacagcgaagggaagcagtatgtcgtggggccgggtctgaaaggggtcagcagcaggggctccaggaggcaggggcactgagcggtacctgggggggaggtggtggggccacacccaggagtcctgtgccccccccactcccgccgttggagatgagaagcaggggccagcctgcgggtccctgagttcagcgcccacccccccgccgcagcaccccggggtctcagcaggctgctgtgctgggggcgggggcgcttatggagccgggagcagccccccccccacggcctcggagcatctctggggcctcagggatggaccggggtctgcaggcaggtgtcctctcgcgcccccactccctgggctataacgtggaagatgcggcccaagcccggtcggtttggcctttgtccccagccagtggggacagcctggccctcaggctgctcgttaagactctaatgacctcgaggcccccagaggcgctgatgacccacggagatgatcccgcaggcctggcagcagggaaatgatccagaaagtgccacctcagcccccagccatctgccacccacctggaggccctcaggggccgggcgccggggggcaggcgctataaagccggctgggcccagccgcccccagccctctgggaccagctgtgttcccaggccaccggcaagcaggtctgtccccctgggctcccgtcagctgggtctgggctgtcctgctggggccagggcatctcggcaggaggacgtgggctcctctctcggagcccttggggggtgaggctggtgggggctgcaggtgcccctggctggcctcaacgccgcccgtcccccaggtcctcaccccccgccatgcatgctgaagggacctttaccagtgatgtaagttcttatttggaaggccaagctgccaaggaattcattgcttggctggtgaaaggccgaggataggatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcatgggggggatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttctttggaaatacaccgacttgattgaagtctcttgaagatagtaaacagtacttacctttgatcccaatgaaatcgagcatttcagttgtaaaagaattccgcctattcataccatgtaatgtaattttacacccccagtgctgacactttggaatatattcaagtaatagactttggcctcaccctcttgtgtactgtattttgtaatagaaaatattttaaactgtgcatatgattattacattatgaaagagacattctgctgatcttcaaatgtaagaaaatgaggagtgcgtgtgcttttataaatacaagtgattgcaaattagtgcaggtgtccttaaaaaaaaaaaaaagtaatataaaaaggaccaggtgttttacaagtgaaatacattcctatttggaaaacagttacatttttatgaagattaccagcgcttgctgactttctaaacataaggctgtattgtcttcctgtaccattgcatttcctcattcccaatttgcacaaggatgtctgggtaaactattcaagaaatggctttgaaatacagcatgggagcttgtctgagttggaatgcagagttgcactgcaaaatgtcaggaaatggatgtctctcagaatgcccaactccaaaggatttatatgtgtatatagtaagcagtttcctgattccagcaggccaaagagtctgctgaatgttgcgttgccggagacctgtatttctcaacaaggtaagatggtatcctagcaactgcggattttaatacattttcagcagaagtacttagttaatctctacctttagggatcgtttcatcatttttagatgttatacttgaaatactgcataacttttagctttcatgggttcctttttttcagcctttaggagactgttaagcaatttgctgtccaacttttgtgttggtcttaaactgcaatagtagtttaccttgtattgaagaaataaagaccatttttatattaaaaaatacttttgtctgtcttcattttgacttgtctgatatccttgcagtgctcattatgtcagttctgtcagatattcacacatcaaaacttaacgtgagctcagtggagttacagctgcggttttgatgctgttattatttctgaaactagaaatgatgttgtcttcatctgctcatcaaacacttcatgcagcagtttaaggctagtgagaaatgcatacatttattgatacttttttaaagtcaactttttatcagatttttttttcatttggaaatatattgttttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccataacttcgtataggatactttatacgaagttatctcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaggcacagtcgaggctgatcagcgagctctagatctgcacccaatcggcaggcacgggcggcgatctccaatctgcgggatcagtcagatcacccgagtgcgtgggcatgacaatcgtgccctggggaccaacacaatccagaagggcctgaatcactgcgaccggccctcccgcgacccagccgagcgagcttagcgaactgtggacgagaactgtgccaccaagcgtaaggccgttctctcgcatttgccttgctaggctcgcgcgagttgctggctgaggcgttctcgaaatcagctcttgttcggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttccggatctatccatctttggtacggggttgtggcaagcttgatatcgaattcctgcaggtcgaaaggcccggagatgaggaagaggagaacagcgcggcagacgtgcgcttttgaagcgtgcgagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgnccagcggggctgctaaagcgcatgctccagactgccttgggaaaagcgcctcccctacccggtagaattcgatgggggatccactagttctagaataacttcgtataggatactttatacgaagttatgcggccgctctagaagtactctcgagaagctttttgaattctttggatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttctttggaaatacaccgacttgattgaagtctcttgaagatagtaaacagtacttacctttgatcccaatgaaatcgagcatttcagttgtaaaagaattccgcctattcataccatgtaatgtaattttacacccccagtgctgacactttggaatatattcaagtaatagactttggcctcaccctcttgtgtactgtattttgtaatagaaaatattttaaactgtgcatatgattattacattatgaaagagacattctgctgatcttcaaatgtaagaaaatgaggagtgcgtgtgcttttataaatacaagtgattgcaaattagtgcaggtgtccttaaaaaaaaaaaaaagtaatataaaaaggaccaggtgttttacaagtgaaatacattcctatttggaaaacagttacatttttatgaagattaccagcgcttgctgactttctaaacataaggctgtattgtcttcctgtaccattgcatttcctcattcccaatttgcacaaggatgtctgggtaaactattcaagaaatggctttgaaatacagcatgggagcttgtctgagttggaatgcagagttgcactgcaaaatgtcaggaaatggatgtctctcagaatgcccaactccaaaggatttatatgtgtatatagtaagcagtttcctgattccagcaggccaaagagtctgctgaatgttgcgttgccggagacctgtatttctcaacaaggtaagatggtatcctagcaactgcggattttaatacattttcagcagaagtacttagttaatctctacctttagggatcgtttcatcatttttagatgttatacttgaaatactgcataacttttagctttcatgggttcctttttttcagcctttaggagactgttaagcaatttgctgtccaacttttgtgttggtcttaaactgcaatagtagtttaccttgtattgaagaaataaagaccatttttatattaaaaaatacttttgtctgtcttcattttgacttgtctgatatccttgcagtgctcattatgtcagttctgtcagatattcacacatcaaaacttaacgtgagctcagtggagttacagctgcggttttgatgctgttattatttctgaaactagaaatgatgttgtcttcatctgctcatcaaacacttcatgcagcagtttaaggctagtgagaaatgcatacatttattgatacttttttaaagtcaactttttatcagatttttttttcatttggaaatatattgttttctaga

SEQ ID NO: 6 - DNA, 99 bp, Sus scrofa
atgcatgctgaagggacctttaccagtgatgtaagttcttatttggaaggccaagctgccaaggaattcattgcttggctggtgaaaggccgaggatag

SEQ ID NO: 7 - protein, 39 aa, Sus scrofa
  MISC_FEATURE (8)..(8): X is an A or G
HDEFERHXEGTFTSDVSSYLEGQAAKEFIAWLVKGRGRR

SEQ ID NO: 8 - protein, 32 aa, Sus scrofa
  MISC_FEATURE (2)..(2): X is an A or G
HXEGTFTSDVSSYLEGGQAAKEFIAWLVKGRG

SEQ ID NO: 9 - DNA, 594 bp, Sus scrofa
gagttcagctgagctggctcccaggtcacctctctgggtcttggtgcccccagcatctcccaggctggccctgccctgggggtgcccttcccaccctgccctgggccttgtggagggcaccctgggctcactgggaggcggtcggccctttccttcccgcaggatgtaagcaccagcctatcttccaggccctgcgccctccctgggtgcccccctcctacctccagagccctgactctaggctcttaggatgtcggtcttggaaaactcctactcatccgtcaagaccctcctgggaaaacccttccttcccagccccccaccctggatctgtgccctttcagcctttgaggccacaaatgaggctgtttccaaaggttggaggcccctgggaagggctgacggccggcctcctcccctccaacccctgggccctgggctctgccctcatccagtctcctgccttgcacaccctctcatagaggcccccagatcttccctggctgcagacgggcctcaggaccccctgctgtcctgggaagccagggcccagctccttcctccgcgtggggtggggcctccccacaggggcct